SEQUENCE LISTING (30 sequences)

SEQ ID NO: 1 - DNA, 1416 bp, Reovirus
  source 1..1416: mol_type = genomic DNA
gctattggtcggatggatcctcgcctacgtgaagaagtagtacggctgataatcgcattaacgagtgataatggagcatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacgttagagcgcacggcggtcactagcgcgggagctcccctctcaatccgtaataaccgtatgaccatgggattaaatgatggactcacgttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggataggcgcaactgagcaaagttacgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 2 - DNA, 1331 bp, Reovirus
  source 1..1331: mol_type = genomic DNA
gctattcgctggtcagttatggctcgcgctgcgttcctattcaagactgttgggtttggtggtctgcaaaatgtgccaattaacgacgaactatcttcacatctactccgagctggtaattcaccatggcagttaacacagtttttagactggataagccttgggaggggtttagctacatcggctctcgttccgacggctgggtcaagatactatcaaatgagttgccttctaagtggcactctccagattccgttccgtcctaaccaccgatggggagacattaggttcttacgcttagtgtggtcagctcctactctcgatggattagtcgtagctccaccacaagttttggctcagcccgctttgcaagcacaggcagatcgagtgtacgactgcgatgattatccatttctagcgcgtgatccaagattcaaacatcgggtgtatcagcaattgagtgctgtaactctacttaacttgacaggttttggcccgatttcctacgttcgagtggatgaagatatgtggagtggagatgtgaaccagcttctcatgaactatttcgggcacacgtttgcagagattgcatacacattgtgtcaagcctcggctaataggccttgggaatatgacggtacatatgctaggatgactcagattgtgttatccttgttctggctatcgtatgtcggtgtaattcatcagcagaatacgtatcggacattctattttcagtgtaatcggcgaggtgacgccgctgaggtgtggattctttcttgttcgttgaaccattccgcacaaattagaccgggtaatcgtagcttattcgttatgccaactagcccagattggaacatggacgtcaatttgatcctgagttcaacgttgacggggtgtttgtgttcgggttcacagctgccactgattgacaataattcagtacctgcagtgtcgcgtaacatccatggctggactggtagagctggtaaccaattgcatgggttccaggtgagacgaatggtgactgaattttgtgacaggttgagacgcgatggtgtcatgacccaagctcagcagaatcaagttgaagcgttggcagatcagactcaacagtttaagagggacaagctcgaaacgtgggcgagagaagacgatcaatataatcaggctcatcccaactccacaatgttccgtacgaaaccatttacgaatgcgcaatggggacgaggtaatacgggggcgactagtgccgcgattgcagcccttatctgatcgtcttggagtgagggggtccccccacacacctcacgactgaccacacattcatc

SEQ ID NO: 3 - DNA, 1198 bp, Reovirus
  source 1..1198: mol_type = genomic DNA
gctaaagtcacgcctgtcgtcgtcactatggcttcctcactcagagctgcgatctccaagatcaagagggatgacgtcggtcagcaagtttgtcctaattatgtcatgctgcggtcctctgtcacaacaaaggtggtacgaaatgtggttgagtatcaaattcgtacgggcggattcttttcgtgcttagctatgctaaggccactccagtacgctaagcgtgagcgtttgcttggtcagaggaatctggaacgtatatcgactagggatatccttcagactcgtgatttacactcactatgtatgccaactcctgatgcgccaatgtctaatcatcaagcatccaccatgagagagctgatttgcagttacttcaaggtcgatcatgcggatgggttgaaatatatacccatggatgagagatactctccgtcatcacttgccagattgtttaccatgggcatggctgggctgcacattaccactgagccatcttataagcgtgttccgattatgcacttagctgcggacttggactgtatgacgctggctctaccttacatgattacgcttgatggtgatactgtggttcctgtcgctccaacactgtcagcggaacagcttctggacgacggactcaaaggattagcatgcatggatatctcctatggatgtgaggtggacgcgaatagccggccggctggtgatcagagtatggactcttcacgctgcatcaacgagttgtattgcgaggagacagcagaagccatctgtgtgcttaagacatgccttgtgttaaattgcatgcagtttaaacttgagatggatgacctagcacataacgctgctgagctggacaagatacagatgatgatacccttcagtgagcgtgtttttaggatggcctcgtcctttgcgactattgatgcccagtgttttaggttttgcgtgatgatgaaggataaaaatctgaaaatagatatgcgtgaaacgacgagactgtggactcgttcagcatcagatgattctgtggccacgtcatctttaagtatttccctggaccggggtcgatgggtggcggctgacgccagtgatgctagactgctggtttttccgattcgcgtgtaatgggtgagtgagctgatgtggtcgccaagacatgtgccggtgtcttggtggtgggtgacgcctaatcatc

SEQ ID NO: 4 - DNA, 1196 bp, Reovirus
  source 1..1196: mol_type = genomic DNA
gctatttttgcctcttcccagacgttgtcgcaatggaggtgtgcttgcccaacggtcatcaggtcgtggacttaattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaaccggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactgaagggagatgctcgtcaaacccaattctccaggacttttgattcgagatcgagtttggaatggggtgtgatggtttatgattactctgagctggatcatgatccatcgaagggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattatccagtgatgattggcgatccgatgattcttggctaaacacccccatcttcacagcgccgggcttgaccaacctggtgtgacgtgggacaggcttcattcatc

SEQ ID NO: 5 - DNA, 2304 bp, Reovirus
  source 1..2304: mol_type = genomic DNA
gctattcgcggtcatggcttacatcgcagttcctgcggtggtggattcacgttcgagtgaggctattggactgctagaatcgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcgaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttcctgtatgtattcttgaaatgttgggtattgagattgcggactattgcattcgtcaagaggatggaatgctcacagattggttcctacttttaaccatgctatctgatggcttgactgatagaaggacgcattgtcaatacttgattaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgttgaactcggatctcattccgcatgatgacaggcttggtcaattatttcaagcgtttttgcctctcgcaaaggacttattagctccaatggccagaaagttttatgataactcaatgagtgagggtagattgctaacattctctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 6 - DNA, 2204 bp, Reovirus
  source 1..2204: mol_type = genomic DNA
ggctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggacacggcaataatcaacacagacaattcatcaggcgccgtgccaagcgaatcagccttggtgccctacatcgatgagccgctggtagtggttacagagcatgctattaccaacttcaccaaagctgagatggcacttgaattcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacagcctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgcgccagttcccgcacccgtgtttgccataccacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgtattagatctagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 7 - DNA, 2241 bp, Reovirus
  source 1..2241: mol_type = genomic DNA
gctaaagtgaccgtggtcatggcttcattcaagggattctccgccaacactgttccagtttctaaggccaagcgtgacatatcatctcttgccgctactcctggacttcgttcacaatccttcactccgtctgtggatatgtctcaatcgcgtgaattcctcacaaaggcaattgagcaagggtccatgtctataccttatcagcatgtgaatgtaccgaaagttgatcgtaaagttgttagcctggtagtgcgacctttctcttcaggtgctttctctatctctggagtgatttcgccagcccatgcctatctactagagtgtctaccccagcttgagcaggcgatggcttttgttgcttcacctgagtctttccaggcttccgacgtcgcgaagcgctttgccataaagccaggtatgagcctccaggatgccatcactgcctttattaactttgtgtccgcgatgctgaaaatgacggtgactcgtcaaaactttgacgttattgtggctgagatcgagaggcttgcttcaaccagcgtgtccgtcaggactgaagaagcgaaggttgctgatgaggagctaatgctattcgggttagatcatagagggccacagcagctggatgtttctgacgctaaagggataatgaaggctgctgatattcagacaactcatgatgtccatttggcaccaggcgttggtaatattgatcctgaaatctataacgaggggcggttcatgttcatgcagcacaagccacttgcggcggatcaatcgtatttcaccttggagactgcggattatttcaagatttatccaacatacgatgaacatgatggcaggatggctgaccaaaagcagtcgggattgatactgtgtactaaggacgaggtattggctgagcaaactatatttaaactggacgcccctgatgacaagactgttcatctgttggatcgcgatgacgaccacgttgttgccagatttactaaggtatttatagaggacgtggctcccgggcatcatgctgctcaaagatcgggacaacgctctgtgcttgatgacctatatgcgaatacgcaagtgatttccattacttctgctgctttaaagtgggtggtcaagcacggcgtatctgatggaatcgtgaacaggaagaatgtcaaagtgtgtgttggttttgaccccctgtacaccttgtctacacataacggggtgtccttatgtgccctgctgatggacgaaaaactctctgtgctgaacagtgcgtgtcgtatgacgttacgctcactcatgaagaccggacgcgacgttgatgcacacagagcttttcagcgagtcctctctcaaggatacacatcgctaatgtgctactatcatccttcacggaagttggcatatggtgaggtgctctttctagaacgatccaatgacgtgacagatgggatcaagcttcagttggacgcatctagacagtgtcatgaatgtcctgtgttgcagcagaaagtggttgagttagagaaacagattattatgcagaagtcaatccagtcagaccctaccccagtggcgctgcaaccattgttgtctcagttgcgtgagttgtctagtgaagttactaggctacagatggagttgagtcgagctcagtccctgaatgctcagttggaggcggatgtcaagtcagctcaatcatgtagcttggatatgtatctgagacaccacacttgcattaatggtcatgctaaagaagatgaattgcttgacgctgtgcgtgtcgcgccggatgtgaggagagaaatcatggaaaagaggagtgaagtgagacaaggttggtgcgaacgtatttctaaggaagcagctgccaaatgtcaaactgttattgatgacctgactttgatgaatggaaagcaagcacaagagataacagaattacgtgattcggctgaaaaatatgagaaacagattgcagagctggtgagtaccatcacccaaaaccagataacgtatcagcaagagctacaagccttggtagcgaaaaatgtggaattggacgcgttgaatcagcgtcaggctaagtctttgcgtattactccctctcttctatcagccactcctatcgattcagttgatgatgttgctgacttaattgatttctctgttccaactgatgagttgtaaataatccgtgatgcagtgttgccctaatcccttaagccttcccgacccccattcatc

SEQ ID NO: 8 - DNA, 3854 bp, Reovirus
  source 1..3854: mol_type = genomic DNA
gctacacgttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggttttctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttatgcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcaaccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaagatgctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcacccgtattcgggaacgcagattatcgcaggtgtttcaatgaacttaaactatatcaaggttattatatggcacaattgcccaggaatcctaagaagtctggacgagcggcccctcgggaggtaagagaacaattcactcaggcattatccgactatctactgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagatattcgagcttttcaaagttattagaggcgtatctgctcgtgaaatggcgaatgtgcgaggcccgcgaaccgtcggtggatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgactctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 9 - DNA, 3916 bp, Reovirus
  source 1..3916: mol_type = genomic DNA
gctaaatggcgcgatggcgaacgtttggggggtgagacttgcagactcgttatcttcacccactattgagacacgaacgcgtcagtataccttacacgatctttgctcagacctagatgctaatccggggagggaaccgtggaaacctctgcgtaatcagcgtactaataatattgtggctgtgcaattattcagaccattgcagggtttagttttagatacccagctttatggatttccaggagcatttgatgactgggagcgattcatgagagagaagctgcgtgtgctaaagtatgaagtattgcgcatctatccaatcagcaactatagcaatgaacatgtcaacgtcttcgtggccaatgctttggtgggcgctttcctgtcgaatcaagctttctatgacctgctaccgttgttgataattaatgacactatgattggtgatctacttggcacgggggcatcgctatcacagttctttcaatctcatggagatgtgctggaagtcgcagctggtcgtaagtatctgcagatggaaaactactccaacgatgacgatgatcctccattatttgcgaaagacctgtcagattatgctaaagcattctacagtgacacatatgaagtgttggacaggttcttttggacgcatgactcttcagcgggggtcttagtgcattatgataagccaacgaatggtcatcactatctgctgggtactttgactcagatggtcagtgcacctccttatattattaacgctactgacgcaatgttgcttgaatcctgtctagaacagttctcagctaatgtgcgtgcgagacctgcgcaacccgttacacgcttagaccaatgctatcatttaagatggggagcacaatatgtaggagaagattcactgacatatcggttgggggtgttatccttgctggctaccaatggatatcaattagctagaccgattccaagacagttgacgaatcgatggttgtcgagctttgtgagtcaaattatgtctgacggcgtcaacgagactccactgtggccccaagaaaggtatgtgcagatcgcttatgattcaccatccgttgttgatggggctacgcaatatggctatgtcaggaagaatcaactcagactcggcatgagaatatcggcgctgcaatcgctgagtgatacgccctcgccggtacagtggcttccacaatacaccatcgaccaggcagcgatggacgaaggcgatctgatggttagtcggcttacgcaactcccgttacgtcctgattatggtaatatctgggtcggcgatgcgctatcctattatgtggactacaatcggagtcatcgagtcgtgctttcatcggaacttcctcagcttccggacacatattttgatggcgatgaacagtatgggcgcagcctgttctcactagctcgtaagattggtgaccgctcgttagtgaaagatacggctgtcttgaagcacgcttaccaagccatcgatccaaatactggtaaggagtatctgagatctcggcaatctgtcgcatattttggtgcatcagcgggtcattctggtgccgaccagccgttagtcatagagccctggattcaagggaaaatcagtggtgtgccgccaccctcctcagtgcgacagttcggctatgatgttgcccgtggcgcgatcgtcgatctggcgagaccatttccttctggagattatcaatttgtctattcggatgttgaccaggtggtcgatggccatgacgatctgagtatatcatctggactggtggagagccttttgtcttcatgcatgcacgccacagcacccgggggctcatttgttgttaagataaattttccgactagacccgtatggcactacatcgaacagaagatcttgcccaatattacgtcatacatgttgatcaagcctttcgtcaccaacaacgtcgaattgttcttcgtcgctttcggtgtgcatcaacactcatcacttacttggacatctggagtgtacttcttcttggtggaccatttttatcgttatgagactttatctacgatctcacgacaattgccgtcttttgggtatgttgatgatgggtcttccgtgactggtatcgagacaattagtattgagaaccctggcttctcgaatatgacccaggccgctcgcattggtatctcaggattgtgtgctaatgtaggtaacgcgcgtaagtccattgccatttacgaatctcatggggccagagtattaactatcacatcaaggagatctccggcatcagctagaagaaagtctaggttgcgatatttgccattaatagaccctaggtcgttagaggtacaggcgcgcactattctgccagctgatccagtgttatttgaaaacgtgagcggagcgtcaccccatgtttgtctgacaatgatgtacaacttcgaagtgtcgtcagcggtatatgatggagacgttgtgctagatcttgggacgggaccagaggctaaaatccttgaactgatacccgcaacctctccagtcacatgcgtggacatacggcctacagcgcagcctagtggatgttggaacgttcgtaccacgttccttgagttagattatttgagcgatggatggatcactggggtgcgtggggacatagttacttgtatgttatctttgggggccgctgccgctggaaaatcaatgacttttgacgctgcgtttcagcaattaatcaaagtattatccaagagtacggctaatgttgtgctggtgcaggttaactgccctacagacgtggtgaggagcattaagggctacctagagatagattcgactaacaagaggtataggttccccaaatttggtcgagacgagccgtactctgacatggatgcgctggagaaaatatgtcgtaccgcctggccaaactgctcaattacctgggttccattgtcatacgacttgcggtggactagactggcattattagagtccacgacattgagtagcgcgtcgattagaattgctgagctgatgtataaatacatgcctattatgaggattgatattcatggactacccatggaaaagcgaggtaacttcatagtggggcagaactgctcattagtaatccctggttttaatgcgcaggatgtctttaactgttatttcaattccgccctcgctttctcgactgaagatgtcaatgctgcgatgattccccaagtgtctgcgcagtttgatgcgactaagggtgagtggacgttggatatggtcttctccgacgcaggaatctataccatgcaggctctagtgggatctaatgctaatccagtctctttgggttcctttgtagttgattctccagatgtagatataactgacgcttggccagctcagttagactttacgatcgcgggaactgatgtcgatataacagttaatccttattaccgtctgatgacctttgtaaggatcgatggacagtggcagattgccaatccagacaaatttcaattcttttcgtcggcgtctgggacgttagtgatgaacgtcaaattagatatcgcagataaatatctactatactatatacgagatgtccagtctcgagatgttggcttttacattcagcatccacttcaacttttgaatacgatcacattgccaaccaacgaggacctttttctgagcgcacctgacatgcgagagtgggcagttaaggaaagcggtaacacgatatgtatactcaatagtcaagggtttgtgctacctcaagattgggatgtgttaacagataccataagttggtccccatcgatacccacatacattgtgccaccgggtgattataccttgactcctctgtaactcactgtccctcgtgagcgcgcctaattcatc

SEQ ID NO: 10 - DNA, 3901 bp, Reovirus
  source 1..3901: mol_type = genomic DNA
gctaatcgtcaggatgaagcggattccaaggaagacaaagggcaaatccagcggaaagggcaatgactcaacagagagagcggacgatggctcgagccaattaagagacaagcaaaacaataaggctggccccgccactacggagcctggcacatccaaccgagagcaatacaaagctcgaccaggtattgcatctgtgcagagggccactgaaagtgcagaaatgcccatgaagaataatgacgaagggacgccagataagaaaggaaatactaagggcgacctagttaatgagcatagtgaggctaaagacgaggcggatgaagcgacgaagaagcaggcaaaggatacagacaaaagtaaagcgcaagtcacatattcagacactggtatcaataatgctaatgaactgtcaagatctgggaatgtggataatgagggtggaagtaatcagaagccgatgtctaccagaatagctgaggcaacgtctgctatagtgtcgaaacatcctgcgcgtgttgggctgccacctaccgctagcagtggtcatgggtatcagtgccatgtctgttctgcagtcctgtttagtcctttagacctagatgcccacgtcgcctcacatggtttgcatggtaacatgacattaacatcgagtgatatccagcgacatataactgagttcatcagctcatggcaaaatcatcctattgttcaagtttcggctgatgtcgaaaataagaaaactgctcaattgcttcacgctgacactcctcgactcgtcacttgggatgctggtttgtgtacttcattcaaaatcgtcccgattgtgccagctcaggtgccgcaggatgtactggcctatacgtttttcacctcttcatacgctatccaatcaccgtttccagaggcggcagtgtctaggattgtggtgcatacgagatgggcatctaatgttgactttgaccgagactcgtctgtcatcatggcgccacctacagaaaacaatatccatttgtttaaacagttactaaatactgaaaccctgtctgtaaggggggctaatccgctaatgttcagggcgaatgtgttgcatatgttgctagagttcgtattagataacttgtatctgaacagacatacgggattctctcaagaccacacgccatttactgagggtgctaatttgcgttcacttcctggccccgatgctgagaaatggtactcgattatgtatccaacgcgcatgggaacgccgaatgtatccaaaatatgtaatttcgtcgcctcttgtgtgcgaaatcgggttggacggtttgatcgagcacagatgatgaacggagctatgtcagagtgggtggatgtcttcgagacttcagacgcgctaaccgtctccattcgaggtcgatggatggctagactagctcgcatgaacataaatccaacagagatcgaatgggcattgactgaatgtgcacaaggatatgtgactgtcacaagtccttacgctcctagcgtaaatagattgatgccctatcgtatctccaacgctgagcggcaaatatcacagataatcaggatcatgaacattggcaataacgcgacggtgatacaacctgttctgcaagatatttcggtactccttcaacgcatatcaccactccaaatagatccaactattatttccaacactatgtcaacagtctcggagtctactactcagaccctcagccccgcgtcctcaattttgggtaaactacgaccaagcaactcagatttttctagttttagagtcgcgttggctggatggctttataatggggttgtgacgacggtgattgatgatagttcatatccaaaagacggcggcagcgtgacctcacttgaaaatctgtgggatttcttcatccttgcgcttgctctaccactgacaactgacccctgtgcacctgtgaaagcattcatgaccctagccaacatgatggttggtttcgagacaatccctatggataatcagatctatactcaatcgagacgcgcgagtgctttctcaacgcctcacacgtggccacgatgctttatgaacatccagttaatttctccaatcgacgctcccatcttgcgacagtgggctgaaattattcatagatactggcctaacccttcacagattcgttatggtgcaccgaacgttttcggctcggcaaatttgttcactccacctgaggtgctgttattgccaatcgatcatcaaccagctaatgtaacaacgccaacgctggacttcaccaatgagttaactaattggcgcgctcgtgtctgtgagcttatgaagaatctcgttgataaccaaagatatcaacctggatggacacaaagtctagtctcgtcaatgcgcggaacgctagacaaattgaagttgattaaatcgatgacaccaatgtatctgcaacagctggctccggtagagttagcagtgatagctcccatgttgccttttccacctttccaggtgccatacgtccgtctcgatcgtgacagagttccaacaatggttggagtaacacgacattcacgagatactattactcagccggcgctatcgctgtcgacaaccaatactactgttggcgtgccactagctctagacgcgagggctatcaccgttgcgctgttgtcagggaaatatccgccggatttggtgacaaatgtatggtacgctgatgccatttacccaatgtatgcagacacggaggtgttctctaatcttcagagagacatgattacctgcgaggccgtgcagacattagtgactctggtggcgcaaatatcagagacccagtatcctgtagataggtatcttgattggatcccatcactgagagcatcggcggcgacggcggcgacatttgctgagtgggttaatacttcaatgaagacggcgtttgatttgtctgatatgctgttagagcctctcctaagcggtgatccgaggatgactcaactagcgattcagtatcagcagtacaatggcagaacgtttaatatcatacctgaaatgccaggttcagtaattgctgactgcgttcaattaacagcagaagtctttaatcacgaatataacctgtttgggattgcgcggggtgatatcatcattggccgtgttcagtcgacacatttgtggtcaccgctggctcctccacctgacctggtgtttgatcgtgatacccctggtgttcacatcttcggacgagattgccgtatatcgtttggaatgaatggcgccgcgccaatgattagagatgagactggactgatggtgccttttgaaggaaattggattttcccactggcgctttggcaaatgaatacacgatattttaatcaacagttcgacgcgtggattaagacaggagagttgcgaatccgcattgagatgggcgcgtatccatatatgttgcattactatgatccacgtcagtacgctaatgcatggaatttaacatccgcctggcttgaagaaattacgccgacgagcatcccatccgtgcctttcatggtgcccatttcaagtgatcatgacatttcctctgccccagctgtccaatatatcatttcaactgaatataatgatcggtctctgttctgcactaactcatcatctccccaaaccatcgctggaccagacaaacacattccagttgagagatataacattctgaccaaccccgacgctccacccacgcagatacaactgcctgaagtcgttgacttgtacaacgtcgtcacacgctatgcgtatgagactccgcctattaccgctgttgttatgggtgttccttgatcctcatcctcccaacaggtgctagagcattgcgctcaatgctagttgggccgattcatc

SEQ ID NO: 11 - protein, 455 aa, Reovirus
MDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 12 - protein, 418 aa, Reovirus
MARAAFLFKTVGFGGLQNVPINDELSSHLLRAGNSPWQLTQFLDWISLGRGLATSALVPTAGSRYYQMSCLLSGTLQIPFRPNHRWGDIRFLRLVWSAPTLDGLVVAPPQVLAQPALQAQADRVYDCDDYPFLARDPRFKHRVYQQLSAVTLLNLTGFGPISYVRVDEDMWSGDVNQLLMNYFGHTFAEIAYTLCQASANRPWEYDGTYARMTQIVLSLFWLSYVGVIHQQNTYRTFYFQCNRRGDAAEVWILSCSLNHSAQIRPGNRSLFVMPTSPDWNMDVNLILSSTLTGCLCSGSQLPLIDNNSVPAVSRNIHGWTGRAGNQLHGFQVRRMVTEFCDRLRRDGVMTQAQQNQVEALADQTQQFKRDKLETWAREDDQYNQAHPNSTMFRTKPFTNAQWGRGNTGATSAAIAALI

SEQ ID NO: 13 - protein, 254 aa, Reovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSNHQASTMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDMDVRWTRIAGRLVIRVWTLHAASTSCIARRQQKPSVCLRHALC

SEQ ID NO: 14 - protein, 366 aa, Reovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSNHQASTMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDISYGCEVDANSRPAGDQSMDSSRCINELYCEETAEAICVLKTCLVLNCMQFKLEMDDLAHNAAELDKIQMMIPFSERVFRMASSFATIDAQCFRFCVMMKDKNLKIDMRETTRLWTRSASDDSVATSSLSISLDRGRWVAADASDARLLVFPIRV

SEQ ID NO: 15 - protein, 365 aa, Reovirus
MEVCLPNGHQVVDLINNAFEGRVSIYSAQEGWDKTISAQPDMMVCGGAVVCMHCLGVVGSLQRKLKHLPHHRCNQQIRHQDYVDVQFADRVTAHWKRGMLSFVAQMHEMMNDVSPDDLDRVRTEGGSLVELNRLQVDPNSMFRSIHSSWTDPLQVVDDLDTKLDQYWTALNLMIDSSDLIPNFMMRDPSHAFNGVKLKGDARQTQFSRTFDSRSSLEWGVMVYDYSELDHDPSKGRAYRKELVTPARDFGHFGLSHYSRATTPILGKMPAVFSGMLTGNCKMYPFIKGTAKLKTVRKLVEAVNHAWGVEKIRYALGPGGMTGWYNRTMQQAPIVLTPAALTMFPDTIKFGDLNYPVMIGDPMILG

SEQ ID NO: 16 - protein, 736 aa, Reovirus
MAYIAVPAVVDSRSSEAIGLLESFGVDAGADANDVSYQDHDYVLDQLQYMLDGYEAGDVIDALVHKNWLHHSVYCLLPPKSQLLEYWKSNPSAIPDNVDRRLRKRLMLKKDLRKDDEYNQLARAFKISDVYAPLISSTTSPMTMIQNLNRGEIVYTTTDRVIGARILLYAPRKYYASTLSFTMTKCIIPFGKEVGRVPHSRFNVGTFPSIATPKCFVMSGVDIESIPNEFIKLFYQRVKSVHANILNDISPQIVSDMINRKRLRVHTPSDRRAAQLMHLPYHVKRGASHVDVYKVDVVDMLFEVVDVADGLRNVSRKLTMHTVPVCILEMLGIEIADYCIRQEDGMLTDWFLLLTMLSDGLTDRRTHCQYLINPSSVPPDVILNISITGFINRHTIDVMPDIYDFVKPIGAVLPKGSFKSTIMRVLDSISILGIQIMPRAHVVDSDEVGEQMEPTFEQAVMEIYKGIAGVDSLDDLIKWVLNSDLIPHDDRLGQLFQAFLPLAKDLLAPMARKFYDNSMSEGRLLTFSHADSELLNANYFGHLLRLKIPYITEVNLMIRKNREGGELFQLVLSYLYKMYATSAQPKWFGSLLRLLICPWLHMEKLIGEADPASTSAEIGWHIPREQLMQDGWCGCEDGFIPYVSIRAPRLVIEELMEKNWGQYHAQVIVTDQLVVGEPRRVSAKAVIKGNHLPVKLVSRFACFTLTAKYEMRLSCGHSTGRGAAYSARLAFRSDLA

SEQ ID NO: 17 - protein, 708 aa, Reovirus
MGNASSIVQTINVTGDGNVFKPSAETSSTAVPSLSLSPGMLNPGGVPWIAVGDETSVTSPGALRRMTSKDIPDTAIINTDNSSGAVPSESALVPYIDEPLVVVTEHAITNFTKAEMALEFNREFLDKMRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDSLIRRYPKEAAVALAKRNGGIQWMDVSEGTVMNEAVNAVAASALAPSASAPPLEEKSKLTEQAMDLVTAAEPEIIASLAPVPAPVFAIPPKPADYNVRTLRIDEATWLRMIPKSMNTPFQIQVTDNTGTNWHLNLRGGTRVVNLDQIAPMRFVLDLGGKSYKETSWDPNGKKVGFIVFQSKIPFELWTAASQIGQATVVNYVQLYAEDSSFTAQSIIATTSLAYNYEPEQLNKTDPEMNYYLLATFIDSAAITPTNMTQPDVWDALLTMSPLSAGEVTVKGAVVSEVVPADLIGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQNWTQGFLDKVSAHFPAPKPDCPTSGDSGESSNRRVKRDSYAGVVKRGYTR

SEQ ID NO: 18 - protein, 721 aa, Reovirus
MASFKGFSANTVPVSKAKRDISSLAATPGLRSQSFTPSVDMSQSREFLTKAIEQGSMSIPYQHVNVPKVDRKVVSLVVRPFSSGAFSISGVISPAHAYLLECLPQLEQAMAFVASPESFQASDVAKRFAIKPGMSLQDAITAFINFVSAMLKMTVTRQNFDVIVAEIERLASTSVSVRTEEAKVADEELMLFGLDHRGPQQLDVSDAKGIMKAADIQTTHDVHLAPGVGNIDPEIYNEGRFMFMQHKPLAADQSYFTLETADYFKIYPTYDEHDGRMADQKQSGLILCTKDEVLAEQTIFKLDAPDDKTVHLLDRDDDHVVARFTKVFIEDVAPGHHAAQRSGQRSVLDDLYANTQVISITSAALKWVVKHGVSDGIVNRKNVKVCVGFDPLYTLSTHNGVSLCALLMDEKLSVLNSACRMTLRSLMKTGRDVDAHRAFQRVLSQGYTSLMCYYHPSRKLAYGEVLFLERSNDVTDGIKLQLDASRQCHECPVLQQKVVELEKQIIMQKSIQSDPTPVALQPLLSQLRELSSEVTRLQMELSRAQSLNAQLEADVKSAQSCSLDMYLRHHTCINGHAKEDELLDAVRVAPDVRREIMEKRSEVRQGWCERISKEAAAKCQTVIDDLTLMNGKQAQEITELRDSAEKYEKQIAELVSTITQNQITYQQELQALVAKNVELDALNQRQAKSLRITPSLLSATPIDSVDDVADLIDFSVPTDEL

SEQ ID NO: 19 - protein, 1267 aa, Reovirus
MSSMILTQFGPFIESISGITDQSNDVFEDAAKAFSMFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVAEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYAWFILKTKSIFPQNTLHSMYESLEGGYCPNLEWLEPRSDYKFMYMGVMPLSAKYARSAPSNDKKARELGEKYGLSSVVGELRKRTKTYVKHDFASVRYIRDAMACTSGIFLVRTPTETVLQEYTQSPEIKVPIPQKDWTGPIGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSTTSGSAVIEKVIPLGVYASSPPNQSINIDISACDASITWDFFLSVIMAAIHEGVASSSIGKPFMGVPASIVNDESVVGVRAARPISGMQNMIQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGTTAGKVNSETIQKMLELISKYGEEFGWKYDIAYDGTAEYLKLYFIFGCRIPNLSRHPIVGKERANSSAEEPWPAILDQIMGVFFNGVHDGLQWQRWIRYSWALCCAFSRQRTMIGESVGYLQYPMWSFVYWGLPLVKAFGSDPWIFSWYMPTGDLGMYSWISLIRPLMTRWMVANGYVTDRCSPVFGNADYRRCFNELKLYQGYYMAQLPRNPKKSGRAAPREVREQFTQALSDYLLQNPELKSRVLRGRSEWEKYGAGIIHNPPSLFDVPHKWYQGAQEAAIATREELAEMDETLMRARRHRYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVNTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 20 - protein, 1289 aa, Reovirus
MANVWGVRLADSLSSPTIETRTRQYTLHDLCSDLDANPGREPWKPLRNQRTNNIVAVQLFRPLQGLVLDTQLYGFPGAFDDWERFMREKLRVLKYEVLRIYPISNYSNEHVNVFVANALVGAFLSNQAFYDLLPLLIINDTMIGDLLGTGASLSQFFQSHGDVLEVAAGRKYLQMENYSNDDDDPPLFAKDLSDYAKAFYSDTYEVLDRFFWTHDSSAGVLVHYDKPTNGHHYLLGTLTQMVSAPPYIINATDAMLLESCLEQFSANVRARPAQPVTRLDQCYHLRWGAQYVGEDSLTYRLGVLSLLATNGYQLARPIPRQLTNRWLSSFVSQIMSDGVNETPLWPQERYVQIAYDSPSVVDGATQYGYVRKNQLRLGMRISALQSLSDTPSPVQWLPQYTIDQAAMDEGDLMVSRLTQLPLRPDYGNIWVGDALSYYVDYNRSHRVVLSSELPQLPDTYFDGDEQYGRSLFSLARKIGDRSLVKDTAVLKHAYQAIDPNTGKEYLRSRQSVAYFGASAGHSGADQPLVIEPWIQGKISGVPPPSSVRQFGYDVARGAIVDLARPFPSGDYQFVYSDVDQVVDGHDDLSISSGLVESLLSSCMHATAPGGSFVVKINFPTRPVWHYIEQKILPNITSYMLIKPFVTNNVELFFVAFGVHQHSSLTWTSGVYFFLVDHFYRYETLSTISRQLPSFGYVDDGSSVTGIETISIENPGFSNMTQAARIGISGLCANVGNARKSIAIYESHGARVLTITSRRSPASARRKSRLRYLPLIDPRSLEVQARTILPADPVLFENVSGASPHVCLTMMYNFEVSSAVYDGDVVLDLGTGPEAKILELIPATSPVTCVDIRPTAQPSGCWNVRTTFLELDYLSDGWITGVRGDIVTCMLSLGAAAAGKSMTFDAAFQQLIKVLSKSTANVVLVQVNCPTDVVRSIKGYLEIDSTNKRYRFPKFGRDEPYSDMDALEKICRTAWPNCSITWVPLSYDLRWTRLALLESTTLSSASIRIAELMYKYMPIMRIDIHGLPMEKRGNFIVGQNCSLVIPGFNAQDVFNCYFNSALAFSTEDVNAAMIPQVSAQFDATKGEWTLDMVFSDAGIYTMQALVGSNANPVSLGSFVVDSPDVDITDAWPAQLDFTIAGTDVDITVNPYYRLMTFVRIDGQWQIANPDKFQFFSSASGTLVMNVKLDIADKYLLYYIRDVQSRDVGFYIQHPLQLLNTITLPTNEDLFLSAPDMREWAVKESGNTICILNSQGFVLPQDWDVLTDTISWSPSIPTYIVPPGDYTLTPL

SEQ ID NO: 21 - protein, 1275 aa, Reovirus
MKRIPRKTKGKSSGKGNDSTERADDGSSQLRDKQNNKAGPATTEPGTSNREQYKARPGIASVQRATESAEMPMKNNDEGTPDKKGNTKGDLVNEHSEAKDEADEATKKQAKDTDKSKAQVTYSDTGINNANELSRSGNVDNEGGSNQKPMSTRIAEATSAIVSKHPARVGLPPTASSGHGYQCHVCSAVLFSPLDLDAHVASHGLHGNMTLTSSDIQRHITEFISSWQNHPIVQVSADVENKKTAQLLHADTPRLVTWDAGLCTSFKIVPIVPAQVPQDVLAYTFFTSSYAIQSPFPEAAVSRIVVHTRWASNVDFDRDSSVIMAPPTENNIHLFKQLLNTETLSVRGANPLMFRANVLHMLLEFVLDNLYLNRHTGFSQDHTPFTEGANLRSLPGPDAEKWYSIMYPTRMGTPNVSKICNFVASCVRNRVGRFDRAQMMNGAMSEWVDVFETSDALTVSIRGRWMARLARMNINPTEIEWALTECAQGYVTVTSPYAPSVNRLMPYRISNAERQISQIIRIMNIGNNATVIQPVLQDISVLLQRISPLQIDPTIISNTMSTVSESTTQTLSPASSILGKLRPSNSDFSSFRVALAGWLYNGVVTTVIDDSSYPKDGGSVTSLENLWDFFILALALPLTTDPCAPVKAFMTLANMMVGFETIPMDNQIYTQSRRASAFSTPHTWPRCFMNIQLISPIDAPILRQWAEIIHRYWPNPSQIRYGAPNVFGSANLFTPPEVLLLPIDHQPANVTTPTLDFTNELTNWRARVCELMKNLVDNQRYQPGWTQSLVSSMRGTLDKLKLIKSMTPMYLQQLAPVELAVIAPMLPFPPFQVPYVRLDRDRVPTMVGVTRHSRDTITQPALSLSTTNTTVGVPLALDARAITVALLSGKYPPDLVTNVWYADAIYPMYADTEVFSNLQRDMITCEAVQTLVTLVAQISETQYPVDRYLDWIPSLRASAATAATFAEWVNTSMKTAFDLSDMLLEPLLSGDPRMTQLAIQYQQYNGRTFNIIPEMPGSVIADCVQLTAEVFNHEYNLFGIARGDIIIGRVQSTHLWSPLAPPPDLVFDRDTPGVHIFGRDCRISFGMNGAAPMIRDETGLMVPFEGNWIFPLALWQMNTRYFNQQFDAWIKTGELRIRIEMGAYPYMLHYYDPRQYANAWNLTSAWLEEITPTSIPSVPFMVPISSDHDISSAPAVQYIISTEYNDRSLFCTNSSSPQTIAGPDKHIPVERYNILTNPDAPPTQIQLPEVVDLYNVVTRYAYETPPITAVVMGVP

SEQ ID NO: 22 - DNA, 3854 bp, Reovirus
  source 1..3854: mol_type = genomic DNA
gctacacgttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggtattctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttattcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcacccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaacgatctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcaaccgtattcgggaacgcagattatcgcaggtgtttcaatgaacttaaactatatcaaggttattatatggcacaattgcccaggaatcctaagaagtctggacgagcggcctctcgggaggtaagagaacaattcactcaggcattatccgactatctaatgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagctattcgagcttttcaaagttattagaggcgtatctgctcgtgaaatggcgaatgtgcgaggcccgcgaaccgtcggttgatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgactctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 23 - protein, 1267 aa, Reovirus
MSSMILTQFGPFIESISGITDQSNDVFEDAAKAFSMFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVAEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYSWFILKTKSIFPQNTLHSMYESLEGGYCPNLEWLEPRSDYKFMYMGVMPLSAKYARSAPSNDKKARELGEKYGLSSVVGELRKRTKTYVKHDFASVRYIRDAMACTSGIFLVRTPTETVLQEYTQSPEIKVPIPQKDWTGPIGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSPTSGSAVIEKVIPLGVYASSPPNQSINIDISACDASITWDFFLSVIMAAIHEGVASSSIGKPFMGVPASIVNDESVVGVRAARPISGMQNMIQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGTTAGKVNSETIQNDLELISKYGEEFGWKYDIAYDGTAEYLKLYFIFGCRIPNLSRHPIVGKERANSSAEEPWPAILDQIMGVFFNGVHDGLQWQRWIRYSWALCCAFSRQRTMIGESVGYLQYPMWSFVYWGLPLVKAFGSDPWIFSWYMPTGDLGMYSWISLIRPLMTRWMVANGYVTDRCSTVFGNADYRRCFNELKLYQGYYMAQLPRNPKKSGRAASREVREQFTQALSDYLMQNPELKSRVLRGRSEWEKYGAGIIHNPPSLFDVPHKWYQGAQEAAIATREELAEMDETLMRARRHSYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVNTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 24 - DNA, 1196 bp, Reovirus
  source 1..1196: mol_type = genomic DNA
gctatttttgcctcttcccagacgttgtcgcaatggaggtgtgcttgcccaacggtcatcaggtcgtggacttgattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaaccggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactggagggagatgctcgtcaaacccaattctccaggacttttgattcgagatcgagtttggaatggggtgtgatggtttatgattactctgagctggagcatgatccatcgaagggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattatccagtgatgattggcgatccgatgattcttggctaaacacccccatcttcacagcgccgggcttgaccaacctggtgtgacgtgggacaggcttcattcatc

SEQ ID NO: 25 - protein, 365 aa, Reovirus
MEVCLPNGHQVVDLINNAFEGRVSIYSAQEGWDKTISAQPDMMVCGGAVVCMHCLGVVGSLQRKLKHLPHHRCNQQIRHQDYVDVQFADRVTAHWKRGMLSFVAQMHEMMNDVSPDDLDRVRTEGGSLVELNRLQVDPNSMFRSIHSSWTDPLQVVDDLDTKLDQYWTALNLMIDSSDLIPNFMMRDPSHAFNGVKLEGDARQTQFSRTFDSRSSLEWGVMVYDYSELEHDPSKGRAYRKELVTPARDFGHFGLSHYSRATTPILGKMPAVFSGMLTGNCKMYPFIKGTAKLKTVRKLVEAVNHAWGVEKIRYALGPGGMTGWYNRTMQQAPIVLTPAALTMFPDTIKFGDLNYPVMIGDPMILG

SEQ ID NO: 26 - DNA, 2203 bp, Reovirus
  source 1..2203: mol_type = genomic DNA
gctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggaaacggcaataatcaacacagacaattcatcaggcgccgtgccaagcgaatcagcgcttgtgccctacatcgatgagccgctggtagtggttacagagcatgctattaccaacttcaccaaagctgagatggcacttgaattcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacacgctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgcgccagttcccgcacccgtgtttgccataccacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgtattagatttagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 27 - protein, 708 aa, Reovirus
MGNASSIVQTINVTGDGNVFKPSAETSSTAVPSLSLSPGMLNPGGVPWIAVGDETSVTSPGALRRMTSKDIPETAIINTDNSSGAVPSESALVPYIDEPLVVVTEHAITNFTKAEMALEFNREFLDKMRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDTLIRRYPKEAAVALAKRNGGIQWMDVSEGTVMNEAVNAVAASALAPSASAPPLEEKSKLTEQAMDLVTAAEPEIIASLAPVPAPVFAIPPKPADYNVRTLRIDEATWLRMIPKSMNTPFQIQVTDNTGTNWHLNLRGGTRVVNLDQIAPMRFVLDLGGKSYKETSWDPNGKKVGFIVFQSKIPFELWTAASQIGQATVVNYVQLYAEDSSFTAQSIIATTSLAYNYEPEQLNKTDPEMNYYLLATFIDSAAITPTNMTQPDVWDALLTMSPLSAGEVTVKGAVVSEVVPADLIGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQNWTQGFLDKVSAHFPAPKPDCPTSGDSGESSNRRVKRDSYAGVVKRGYTR

SEQ ID NO: 28 - DNA, 2304 bp, Reovirus
  source 1..2304: mol_type = genomic DNA
gctattcgcggtcatggcttacatcgcagttcctgcggtggtggattcacgttcgagtgaggctattggactgctagaatcgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcgaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttcctgtatgtattcttgaaatgttgggtattgagattgcggactattgcattcgtcaagaggatggaatgctcacagattggttcctacttttaaccatgctatctgatggcttgactgatagaaggacgcattgtcaatacttgatgaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgttgaactcggatctcattccgcatgatgacaggcttggtcaattatttcaagcgtttttgcctctcgcaaaggacttattagctccaatggccagaaagttttatgataactcaatgagtgagggtagattgctaacattcgctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 29 - protein, 736 aa, Reovirus
MAYIAVPAVVDSRSSEAIGLLESFGVDAGADANDVSYQDHDYVLDQLQYMLDGYEAGDVIDALVHKNWLHHSVYCLLPPKSQLLEYWKSNPSAIPDNVDRRLRKRLMLKKDLRKDDEYNQLARAFKISDVYAPLISSTTSPMTMIQNLNRGEIVYTTTDRVIGARILLYAPRKYYASTLSFTMTKCIIPFGKEVGRVPHSRFNVGTFPSIATPKCFVMSGVDIESIPNEFIKLFYQRVKSVHANILNDISPQIVSDMINRKRLRVHTPSDRRAAQLMHLPYHVKRGASHVDVYKVDVVDMLFEVVDVADGLRNVSRKLTMHTVPVCILEMLGIEIADYCIRQEDGMLTDWFLLLTMLSDGLTDRRTHCQYLMNPSSVPPDVILNISITGFINRHTIDVMPDIYDFVKPIGAVLPKGSFKSTIMRVLDSISILGIQIMPRAHVVDSDEVGEQMEPTFEQAVMEIYKGIAGVDSLDDLIKWVLNSDLIPHDDRLGQLFQAFLPLAKDLLAPMARKFYDNSMSEGRLLTFAHADSELLNANYFGHLLRLKIPYITEVNLMIRKNREGGELFQLVLSYLYKMYATSAQPKWFGSLLRLLICPWLHMEKLIGEADPASTSAEIGWHIPREQLMQDGWCGCEDGFIPYVSIRAPRLVIEELMEKNWGQYHAQVIVTDQLVVGEPRRVSAKAVIKGNHLPVKLVSRFACFTLTAKYEMRLSCGHSTGRGAAYSARLAFRSDLA

SEQ ID NO: 30 - protein, 418 aa, Reovirus; identical to SEQ ID NO: 12

(19) United States
(12) Patent Application Publication
(10) Pub. No.: US 2025/0346868 A1
(43) Pub. Date: Nov. 13, 2025

(54) Viral Extraction from Cell Culture

(71) Applicant: National Research Council of Canada, Ottawa (CA)

(72) Inventors: Hue Tran, Calgary (CA); Matthew Coffey, Calgary (CA); Allison Hagerman, Calgary (CA); Robert Voyer, Lachine (CA); Krishna Raj Tiwari, Mont-Royal (CA); Jyoti Latawa, Mont-Royal (CA); Mehul Patel, Dollard Des Ormeaux (CA); Allan Matte, Dollard Des Ormeaux (CA)

(73) Assignee: National Research Council of Canada, Ottawa, ON (CA)

(21) Appl. No.: 19/273,808

(22) Filed: Jul. 18, 2025

Related U.S. Application Data
(63) Continuation of application No. PCT/IB2024/053862, filed on Apr. 19, 2024.
(60) Provisional application No. 63/461,216, filed on Apr. 21, 2023.

(51) Int. Cl. C12N 7/00

(52) U.S. Cl. CPC C12N 7/00; C12N 2720/12251

(57) ABSTRACT

Provided herein are methods of producing virus from a culture of host cells. The methods include providing a culture of host cells which has been infected by the virus; contacting the host cells with a first buffer comprising a detergent and incubating the host cells in the presence of the first buffer for a first period of time thereby producing a cell lysate; contacting the cell lysate with a second buffer comprising an endonuclease for a second period of time to degrade the host cell nucleic acids; and collecting the virus.

### CROSS-REFERENCE TO RELATED APPLICATION(S)

This application is a bypass continuation of International Application No. PCT/IB2024/053862, filed Apr. 19, 2024, which claims the benefit of priority of U.S. Provisional Application No. 63/461,216, filed Apr. 21, 2023, the contents of each of which are herein incorporated by reference in their entireties.

### REFERENCE TO AN ELECTRONIC SEQUENCE LISTING

The contents of the electronic sequence listing (2024-013-02_SL.xml; Size: 71,043 bytes; and Date of Creation: Apr. 16, 2024) is herein incorporated by reference in its entirety.

### BACKGROUND

Triton X-100 was added to Registration, Evaluation, Authorization and restriction of chemicals (REACH) list in December of 2012 and the REACH Annex XIV sunset date was Jan. 2, 2021. After this date, Triton X-100 cannot be used for commercial production in the European Union due to its environmental impact. Therefore, alternative detergent needs to be identified to replace the use of Triton X-100 in the manufacture of viral drug products.

### BRIEF SUMMARY

Provided herein are methods of producing virus from a culture of host cells. The methods include providing a culture of host cells which has been infected by the virus; contacting the host cells with a first buffer comprising a detergent and incubating the host cells in the presence of the first buffer for a first period of time thereby producing a cell lysate; contacting the cell lysate with a second buffer comprising an endonuclease for a second period of time to degrade the host cell nucleic acids; and collecting the virus.

### BRIEF DESCRIPTION OF THE DRAWINGS

FIG. 1 is a graph showing post-clarification residual host cell (HC) DNA results of testing increasing Benzonase® concentrations from 10 to 20 U/mL and for various durations between 60 to 240 minutes with a cell lysate obtained after a 2 hour incubation of a virus containing cell culture with 1% Tween® 20 and with 20 mM Na₂HPO₄, pH 8.0. FIG. 1 shows increasing the Benzonase concentration lowered the residual HC DNA levels to those obtained with Triton X-100 cell lysis.

FIG. 2 is a graph showing residual HC DNA results of the same testing materials as in FIG. 1, after one overnight (O/N) storage at 2-8° C. post-clarification. FIG. 2 shows increasing the Benzonase concentration lowered the residual HC DNA levels to those obtained with Triton X-100 cell lysis for Benzonase treatment of at least 90 minutes before clarification, for the same extended storage at 2-8° C. post-clarification.

FIG. 3 is a graph showing residual HC DNA results of the same testing materials as in FIG. 1, after two overnight storages at 2-8° C. post-clarification. FIG. 3 shows increasing the Benzonase concentration lowered the residual HC DNA levels to those obtained with Triton X-100 cell lysis for the same extended storage at 2-8° C. post-clarification.

FIG. 4 is a graph showing total viral particle titer results for the same testing materials as in FIG. 1 (post-clarification; solid lines), as in FIG. 2 (1×O/N storage at 2-8° C. post-clarification; dashed lines), and as in FIG. 3 (2×O/N storage at 2-8° C. post-clarification; dotted lines). Results of testing material lysed with 1% Triton X-100 for 1 hour incubation and testing material lysed using 1% of QL300 grade Tween 20 in 20 mM Na₂HPO₄ pH 8.0 lysis buffer for 2 hour incubation, both treated with 10 U/mL of Benzonase for 60 minutes are included as controls. Increasing Benzonase concentration from 10 to 20 U/mL or changing Benzonase treatment duration from 60 to 240 minutes did not negatively impact the total reovirus titer (HPLC), for up to 2 days of overnight storage at 2-8° C. before freezing at −80° C. QL: Quality Level.

FIG. 5 is a schematic showing the extraction and purification protocol using Triton X-100 (left side) and the extraction and purification protocol described in the present application (right side).

### DETAILED DESCRIPTION

Due to the vast number of diseases caused by viruses, virology has been an intensively studied field. There has always been the demand to produce viruses efficiently in order to isolate and purify viral proteins, to generate vaccines, or to provide infectious viruses for laboratory studies. Recently, the new development of virus therapy has further necessitated the need for efficient production of infectious viruses.

Described herein is an improved method for the extraction of viruses from cell culture using detergent conditions that can be applied to both small- and large-scale virus production. The method involves an extraction step in which a detergent is added to the cell culture. Thereafter, cell debris can be removed from the extraction mixture by, for example, filtration or centrifugation. The resulting virus suspension can be further concentrated and/or enriched by chromatographic methods. The virus prepared according to the present invention can be used for any purpose, including purification of viral proteins, vaccination, infection of host cells and clinical administration. The methods provided herein are advantageous for the production of viral drug substances, as they result in overall higher yields and the detergents provided herein are a superior replacement to Triton X-100. The methods also result in decreased amounts of residual host cell DNA (HC DNA).

As used herein, “adherent cells” refer to cells which adhere to the culture containers in a cell culture. Examples of adherent cells include monolayer cells, which are cells that form a single layer of cells on the surface of a culture container. “Suspension cells” or “suspended cells” refer to cells which do not adhere to culture containers in a cell culture. Suspension cells can be grown in a “spin culture,” which is a culture in which the culture medium is stirred continuously during the culture process.

As used herein, “ambient temperature” refers to a temperature between about 10° C. and about 30° C. Ambient temperature is preferably between about 15° C. and about 30° C., more preferably between about 20° C. and about 25° C., and most preferably about 25° C.

As used herein, a virus that is “cell associated” refers to a virus which is attached to or trapped in part of a cell in which the virus has been produced. Thus, a virus is cell associated before the host cell is lysed. When cell lysis begins, a virus may be still attached to or trapped in part of the broken cell and remain cell associated. However, when the virus is released free into the medium, it is not cell associated anymore. A “cell free virus” is a virus which is not cell associated.

As used herein, a “cell culture” or “culture of cells” means a population of cultured cells as found in their culture conditions. In particular, a cell culture includes the cells and the culture medium. Cells that have been pelleted are not considered a cell culture unless they are placed in culture medium under culture conditions again.

As used herein, “cell lysis” refers to the disruption of the cell membrane of a cell and the subsequent release of all or part of the content of the cell.

As used herein, “clinical administration” of a substance refers to contacting any part of the body of a living organism with the substance in order to improve or maintain the organism's health conditions.

As used herein, “collecting” the virus refers to the act of separating the virus produced from a cell culture which has been previously infected with the virus. The virus is typically collected by separating cellular debris from the virus and harvesting the portion which comprises the virus. Optionally, the virus can be further separated from the soluble substances, e.g., by centrifugation.

As used herein, “culture conditions” refer to the conditions used in a cell culture, including but not limited to the temperature, type of culture containers, humidity, concentration of CO2 or any other gas used in the culture containers, type of culture medium, the initial density of the cultured cells, and, if the cells are infected with a virus, the initial multiplicity of infection.

As used herein, “cytopathic effect” is the damage to infected host cells. Cytopathic effect may be indicated by cells becoming swollen and granular in appearance and cell clumps breaking up. Cells which show a cytopathic effect may also take up the staining dye in a viable cell count.

As used herein, a “detergent” is a substance having a hydrophilic moiety and a hydrophobic moiety. The detergent is preferably a synthetic chemical compound and more preferably a biodegradable synthetic chemical compound. A detergent useful in the present invention enhances disruption of cell membranes to facilitate release of the content of the disrupted cells.

As used herein, a cell is “disrupted” when the cell membrane is ruptured and at least some of the cell content is released from the cell. A cell may be disrupted, for example, by freeze-thawing, sonication or detergent treatments.

As used herein, “extracting” a virus refers to the act of converting a cell associated virus into a cell free virus.

As used herein, “HEK 293 cells” refer to the human embryo kidney cell line designated 293 (ATCC Number CRL-1573) or its derivatives. For example, 293/SF cells (ATCC Number CRL-1573.1) are HEK 293 cells which have been adapted to grow in serum-free media. Also contemplated in this invention are HEK 293 cells adapted to grow in other culture conditions, or any kind of HEK 293 cells or derivatives which are transformed with an exogenous DNA, provided that this transformation does not impair the ability of the cells to support efficient reovirus production as described in this invention.

As used herein, “incubating” after addition of a detergent to a cell culture refers to the act of allowing the cell culture to be mixed with the detergent for a period of time.

As used herein, “multiplicity of infection” or “MOI” refer to the ratio of the number of virus to the number of cells when a virus is used to contact cells.

As used herein, a “non-enveloped virus” is a virus which does not have an envelope. For example, a non-enveloped virus may be any virus which belongs to the family of Adenoviridae (e.g., adenovirus), Picornaviridae (e.g., polio virus), Reoviridae (e.g., reovirus), Papovarviridae (e.g., papilloma virus), Parvoviridae (e.g., Kilham rat virus) or Iridoviridae (e.g., tipula iridescent virus).

As used herein, “viability of the cells” or “percentage of cells remaining viable” is the percentage of the cells which do not show a cytopathic effect in a population.

As used herein, “viral infection” refers to the entry of a virus into a cell and the subsequent replication of the virus in the cell.

The term “about”, as used herein, may be used to take into account experimental error, measurement error, and variations that would be expected by a person having ordinary skill in the art. For example, “about” may mean plus or minus 10%, or plus or minus 5%, of the indicated value to which reference is being made.

Provided herein is a method of producing virus from a culture of host cells. The methods include providing a culture of host cells which has been infected by the virus and contacting the host cells with a first buffer comprising a detergent and incubating the host cells in the presence of the first buffer for a first period of time thereby producing a cell lysate. The cell lysate is then contacted by a second buffer comprising an endonuclease for a second period of time to degrade the host cell nucleic acids followed by collection of the virus.

The detergent can be a nonionic detergent or an anionic detergent. The nonionic detergent can be Tween® 20 (polysorbate 20), Octyl Beta-D-Glucopyranoside (OGP), Tergitol™ 15-S-9 (C12-14 secondary alcohol ethoxylate, also referred to as polyethylene glycol trimethylnonyl ether) or Tween 80 (polysorbate 80). The anionic detergent can be sodium deoxycholate. The detergents can be present at a concentration of between 0.5% to 2.0% (v/v) or any amount in between 0.5% and 2.0% (v/v). Thus, for example, the detergent can be at a concentration of 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% (v/v) in the buffer. Optionally, the detergent is Tween 20 and is present in the buffer at a concentration of 0.5%, 1.0% or 2.0% (v/v).

In the provided methods, the first buffer containing the detergent can also include a phosphate. The first buffer can contain phosphate, for example, at a concentration of 10 mM to 25 mM phosphate, 10 mM to 20 mM phosphate, 15 mM to 25 mM phosphate, 10 mM to 15 mM phosphate, 15 mM to 20 mM phosphate or any concentration between 10 mM and 25 mM phosphate. Optionally, the first buffer contains phosphate at a concentration of at least 20 mM phosphate. The phosphate can be, for example, sodium phosphate or potassium phosphate.

In the provided methods, the host cells can be incubated in the presence of the first buffer for a period of time. Optionally, the first period of time is 60 minutes, 120 minutes, 180 minutes or 240 minutes. In addition, incubating the host cells in the presence of the first buffer can occur at a pH of 7.0 to 8.0. Optionally, incubating the host cells in the presence of the first buffer occurs at a pH of 8.0. Incubation of the host cells can occur at a particular temperature. For example, incubating the host cells can occur at a temperature of 30° C. to 40° C. Optionally, incubating the host cells occurs at a temperature of 35° C. to 39° C. Optionally, incubating the host cells occurs at a temperature of 36° C. to 38° C. Optionally, incubating the host cells occurs at a temperature of 37° C.

In the provided methods, contacting of the host cells with the first buffer can occur under agitation conditions. For example, contacting the host cells with the first buffer can occur under agitation at 120 rpm.

In the provided methods, the cell lysate is then contacted by a second buffer comprising an endonuclease for a second period of time to degrade the host cell nucleic acids. Optionally, the second period of time is 60 minutes, 90 minutes, 120 minutes, 180 minutes, or 240 minutes. Optionally, the endonuclease is Benzonase (a promiscuous endonuclease that cleaves all forms of DNA and RNA, including single-stranded, double-stranded, linear, and circular) or DNase I (an endonuclease that cleaves single- and double-stranded DNA). The endonuclease can be contacted with the cell lysate at a concentration of 10 to 20 U/mL or any amount between 10 and 20 U/mL. For example, the concentration of the endonuclease can be 10, 11, 12, 13, 14, 15, 16, 17, 18, 19, or 20 U/mL. The cell lysate can be contacted with the endonuclease at a particular temperature. Optionally, contacting the cell lysate with the endonuclease occurs at a temperature of 30° C. to 40° C. Optionally, contacting the cell lysate with the endonuclease occurs at a temperature of 35° C. to 39° C. Optionally, contacting the cell lysate with the endonuclease occurs at a temperature of 37° C. Optionally, the contacting of the cell lysate with the endonuclease can occur under agitation conditions. For example, the contacting of the cell lysate with the endonuclease can be at 120 rpm.

In the provided methods, the second buffer can include other components. For example, the second buffer can include MgCl₂.

The methods provided herein can further include removing cell debris. Optionally, the cell debris is removed by filtration. Thus, the virus can be purified based on, for example, the size or density difference between the virus and the other constituents in the extract. Particularly, filtration or centrifugation can be employed to separate cell debris from the virus. The cell debris can be removed in a single step or using a step-wise filtration protocol. For example, a pre-filter having a relatively large pore size (e.g., 5 μm or 8 μm) can be first used to remove large pieces from the extraction mixture, followed by filters with small pore sizes, such as a combination filter unit containing a 3 μm filter and a 0.8 μm filter. Optionally, after the 5 μm or 8 μm pre-filter step, a filter having a single pore size of 0.8 μm can be used.

In the provided methods, the cells can be mammalian cells. Optionally, the cells are human embryo kidney 293 (HEK 293) cells. Other cells suitable for use in the provided methods include, but are not limited to, mouse L929 cells, African green monkey kidney cells (Vero cells) and Chinese hamster ovary (CHO) cells. The mammalian cells are, optionally, grown in suspension.

The virus can be purified using any means suitable for viral purification. For example, the virus can be purified based on its surface charge. Optionally, the virus can be purified by ion exchange chromatography or by size exclusion chromatography. Optionally, the methods further include purifying the virus by a combination of ion exchange and size exclusion chromatography. Optionally, the ion exchange is performed prior to the size exclusion chromatography. Optionally, the ion exchange is performed after the size exclusion chromatography. The ion exchange can be performed using an anion exchanger. Optionally, a phosphate buffer can be used in the ion exchange and can be, for example, at a concentration of 100 mM sodium phosphate or monosodium phosphate. Optionally, a phosphate buffer is used in the size exclusion chromatography and can be, for example, at a concentration of between 10-15 mM sodium phosphate, e.g., 10, 11, 12, 13, 14, or 15 mM sodium phosphate. Optionally, the size exclusion chromatography is carried out at a pH of 7.0 to 7.5, for example, pH 7.0, 7.1, 7.2, 7.3, 7.4 or 7.5.

The virus may also be purified based on the difference in size, for example, with size exclusion chromatography. Optionally, an anion exchange column can be used prior to size exclusion chromatography. Other chromatographic methods, such as those based on affinity or hydrophobic interaction, can also be used where appropriate.

Also provided herein is a method of producing reovirus that includes providing a culture of HEK 293 cells which has been infected by reovirus; contacting the cells with a first buffer comprising Tween 20 and a phosphate and incubating the HEK 293 cells in the presence of the first buffer for a first period of time thereby producing a cell lysate, wherein the incubating occurs at a temperature of about 30° C. to about 40° C. and a pH of about 7.0 to about 8.0; contacting the cell lysate with a second buffer comprising an endonuclease for a second period of time to degrade the host cell nucleic acids; removing cell debris by filtration; concentrating the filtrate by ultrafiltration and diafiltration; purifying the reovirus by a combination of ion exchange and size exclusion chromatography; and collecting the reovirus. Optionally, the first buffer comprises 15 mM to 25 mM phosphate. Optionally, the first buffer comprises at least 20 mM phosphate. The phosphate can be, for example, sodium phosphate. Optionally, the first buffer comprises 0.5%, 1.0% or 2.0% (v/v) Tween 20. The first period of time can be 60 minutes, 120 minutes, 180 minutes or 240 minutes. The second period of time can be 60 minutes, 90 minutes, 120 minutes, 180 minutes, or 240 minutes. The endonuclease can be Benzonase. The endonuclease can be contacted with the cell lysate at a concentration of 10 to 20 U/mL. Optionally, contacting the cell lysate with the endonuclease occurs at a temperature of 30° C. to 40° C. Optionally, contacting the cell lysate with the endonuclease occurs at a temperature of 35° C. to 39° C. Optionally, contacting the cell lysate with the endonuclease occurs at a temperature of 37° C. Optionally, incubating the host cells occurs at a temperature of 35° C. to 39° C. Optionally, incubating the host cells occurs at a temperature of 37° C. Optionally, incubating the host cells in the presence of the first buffer occurs at a pH of 7.0 to 8.0, or at a pH of 8.0. In these methods, the second buffer can further include MgCl₂.

Oncolytic viruses that are used in the provided methods and kits include, but are not limited to, oncolytic viruses that are members in the family of reoviridae, myoviridae, siphoviridae, podoviridae, tectiviridae, corticoviridae, plasmaviridae, lipothrixviridae, fuselloviridae, poxyiridae, iridoviridae, phycodnaviridae, baculoviridae, herpesviridae, adenoviridae, papovaviridae, polydnaviridae, inoviridae, microviridae, geminiviridae, circoviridae, parvoviridae, hepadnaviridae, retroviridae, cystoviridae, birnaviridae, paramyxoviridae, rhabdoviridae, filoviridae, orthomyxoviridae, bunyaviridae, arenaviridae, leviviridae, picornaviridae, sequiviridae, comoviridae, potyviridae, caliciviridae, astroviridae, nodaviridae, tetraviridae, tombusviridae, coronaviridae, flaviviridae, togaviridae, and barnaviridae. Immunoprotected viruses and reassortant or recombinant viruses of these and other oncolytic viruses are also encompassed by the provided methods. Thus, the oncolytic virus used in the provided methods is, for example, selected from the group consisting of a reovirus, a Newcastle disease virus (NDV), a vesicular stomatitis virus (VSV), an adenovirus, a vaccinia virus, a parapox orf virus, a Sindbis virus, and a herpes simplex virus. Furthermore, a combination of at least two oncolytic viruses can also be employed to practice the provided methods. A few oncolytic viruses are discussed below, and a person of ordinary skill in the art can practice the present methods using other oncolytic viruses as well according to the disclosure herein and knowledge available in the art.

The virus used in the provided methods and kits may be a non-enveloped virus. Optionally, the virus is a reovirus, for example, a mammalian reovirus, a human reovirus, a serotype 3 virus, a serotype 3 reovirus of the Dearing strain or a recombinant reovirus.

When a virus enters a cell, double-stranded RNA kinase (PKR) is activated, blocking protein synthesis, and the virus cannot replicate in this cell. Some viruses have developed a system to inhibit PKR and to facilitate viral protein synthesis as well as viral replication. For example, adenovirus makes a large amount of a small RNA, VA1 RNA. VA1 RNA has extensive secondary structures and binds to PKR in competition with the double-stranded RNA (dsRNA) that normally activates PKR. Since a minimum length of dsRNA is required to activate PKR, VA1 RNA does not activate PKR. Instead, it sequesters PKR by virtue of its large amount. Consequently, protein synthesis is not blocked, and adenovirus can replicate in the cell. Accordingly, if the PKR inhibitors in adenovirus, vaccinia virus, herpes simplex virus, or parapox orf virus are mutated so as not to block PKR function anymore, the resulting viruses do not infect normal cells due to protein synthesis inhibition by PKR, but they replicate in cancer cells lacking PKR activities. Optionally, the oncolytic virus is an adenovirus mutated in the VA1 region, a vaccinia virus mutated in the K3L and/or E3L region, a vaccinia virus mutated in the thymidine kinase (TK) gene, a vaccinia virus mutated in the vaccinia growth factor (VGF) gene, a herpes virus mutated in the γ134.5 gene, a parapox orf virus mutated in the OV20.0 L gene, or an influenza virus mutated in the NS-1 gene.

Vaccinia viruses mutated in the viral thymidine kinase (TK) gene are unable to make nucleotides needed for DNA replication. In normal cells, the cellular TK levels are usually very low and the virus is unable to replicate. In tumors, loss of the tumor suppressor Rb or an increase in cyclin activity leads to E2F pathway activation and high levels of TK expression. Thus, cancer cells have high TK levels and the mutated vaccinia virus can replicate and spread.

The vaccinia growth factor (VGF) gene is a homolog of mammalian epidermal growth factor (EGF) and can bind and activate the EGF Receptor (EGFR). Vaccinia viruses mutated in the VGF gene are growth restricted to cells with activated EGF pathways, which is commonly mutated in cancers.

The viruses can be modified or mutated according to the known structure-function relationship of the viral PKR inhibitors. For example, since the amino terminal region of E3 protein interacts with the carboxy-terminal region domain of PKR, deletion or point mutation of the carboxy-terminal region domain prevents anti-PKR function (Chang et al., PNAS 89:4825-4829 (1992); Chang et al., Virology 194:537-547 (1993); Chang et al., J. Virol. 69:6605-6608 (1995); Sharp et al., Virol. 250:301-315 (1998); and Romano et al., Mol. and Cell. Bio. 18:7304-7316 (1998)). The K3L gene of vaccinia virus encodes pK3, a pseudosubstrate of PKR. Truncations or point mutations within the C-terminal portion of K3L protein that is homologous to residues 79 to 83 in eIF-2 abolish PKR inhibitory activity (Kawagishi-Kobayashi et al., Mol. Cell. Biology 17:4146-4158 (1997)).

Another example is the Delta24 virus, which is a mutant adenovirus carrying a 24 base pair deletion in the ElA region. (Fueyo et al., Oncogene 19 (1): 2-12 (2000)). This region is responsible for binding to the cellular tumor suppressor Rb and inhibiting Rb function, thereby allowing the cellular proliferative machinery, and hence virus replication, to proceed in an uncontrolled fashion. Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell.

In addition, vesicular stomatitis virus (VSV) selectively kills neoplastic cells. A herpes simplex virus 1 (HSV-1) mutant defective in ribonucleotide reductase expression, hrR3, replicates in colon carcinoma cells but not normal liver cells (Yoon et al., FASEB J. 14:301-311(2000)). Newcastle disease virus (NDV) replicates preferentially in malignant cells, and the most commonly used strain is 73-T (Reichard et al., J. Surgical Research 52:448-453 (1992); Zorn et al., Cancer Biotherapy 9 (3): 22-235 (1994); Bar-Eli et al., J. Cancer Res. Clin. Oncol. 122:409-415 (1996)). Vaccinia virus propagates in several malignant tumor cell lines. Encephalitis virus has an oncolytic effect in a mouse sarcoma tumor, but attenuation may be required to reduce its infectivity in normal cells. Tumor regression has been described in tumor patients infected with herpes zoster, hepatitis virus, influenza, varicella, and measles virus (for a review, see Nemunaitis, J. Invest. New Drugs 17:375-386 (1999)).

Optionally, the oncolytic virus is a modified, non-reovirus virus comprising a reovirus sigma 1 protein, wherein the reovirus sigma 1 protein replaces the native attachment protein of the non-reovirus virus and wherein the modified virus does not comprise any portion of the native attachment protein of the non-reovirus virus. In the modified, non-reovirus virus, the reovirus sigma 1 protein attaches to carrier cells that protect the virus from neutralizing antibodies during in vivo delivery to a tumor, for example, during systemic delivery. The non-reovirus virus can be, but is not limited to, an adenovirus, a vaccinia virus, a herpes simplex virus, a Sindbis virus, or a parapox virus. Optionally, the full-length sequence of the native attachment protein of the non-reovirus virus is replaced with a reovirus sigma 1 protein. Replacement of the native attachment protein of the virus with a reovirus sigma 1 protein allows the virus to attach to carrier cells which protect the virus from neutralizing antibodies during in vivo delivery. The reovirus sigma-1 protein is described in, for example, WO 2008/11004, which is incorporated by reference herein in its entirety.

Optionally, the oncolytic virus is a reovirus. Reovirus refers to any virus classified in the reovirus genus, whether naturally occurring, modified, or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reoviruses have been recovered from many species. Thus, the reovirus can be a mammalian reovirus or a human reovirus. All three types share a common complement-fixing antigen.

Human reovirus includes three serotypes: type 1 (strain Lang or T1L), type 2 (strain Jones, T2J), and type 3 (strain Dearing or strain Abney, T3D). The three serotypes are easily identifiable on the basis of neutralization and hemagglutinin-inhibition assays. A reovirus according to this disclosure can be a type 3 mammalian orthoreovirus. Type 3 mammalian orthoreoviruses include, without limitation, Dearing and Abney strains (T3D or T3A, respectively). See, for example, ATCC Accession Nos. VR-232 and VR-824. See, for example, U.S. Pat. Nos. 6,110,461; 6,136,307; 6,261,555; 6,344,195; 6,576,234; and 6,811,775, which are incorporated by reference herein in their entireties.

Optionally, the provided methods include the use of reoviruses with mutations. For example, mutant reoviruses as described herein can contain a mutation that reduces or essentially eliminates expression of a sigma3 polypeptide or that results in the absence of a functional sigma3 polypeptide as described in U.S. Publication No. 2008/0292594, which is incorporated by reference herein in its entirety. A mutation that eliminates expression of a sigma3 polypeptide or that results in the absence of a functional sigma3 polypeptide can be in the nucleic acid encoding the sigma3 polypeptide (i.e., the S4 gene) or in a nucleic acid that encodes a polypeptide that regulates the expression or function of the sigma3 polypeptide.

As used herein, a mutation that reduces the expression of a sigma3 polypeptide refers to a mutation that results in a decrease in the amount of sigma3 polypeptide, compared to a reovirus expressing wild type levels of sigma3 polypeptide, of at least 30% (e.g., at least 40%, at least 50%, at least 60%, at least 70%, at least 80%, at least 90%, or at least 95%). As used herein, a mutation that essentially eliminates expression of a sigma3 polypeptide refers to a mutation that results in a decrease in the amount of sigma3 polypeptides, relative to the amount of sigma3 polypeptides produced by a wild type reovirus, of at least 95% (e.g., at least 96%, at least 97%, at least 98%, at least 99%, or 100%). As used herein, a mutation that results in a decrease in or absence of a functional sigma3 polypeptide refers to a mutation that allows expression of the sigma3 polypeptide but that results in a sigma3 polypeptide that is not able to assemble or incorporate into the viral capsid. It would be understood that it may be desirable or necessary for sigma3 polypeptides to retain other functionalities (e.g., the ability to bind RNA) in order for the mutant reovirus to retain the ability to propagate.

A mutation in a sigma3 polypeptide as described herein can result in a sigma3 polypeptide that is incorporated into the capsid at levels that are reduced relative to a sigma3 polypeptide that does not contain the mutation (e.g., a wild type sigma3 polypeptide). A mutation in a sigma3 polypeptide as described herein also can result in a sigma3 polypeptide that cannot be incorporated into a viral capsid. Without being bound by any particular mechanism, a sigma3 polypeptide may have reduced function or lack function due, for example, to an inability of the sigma3 polypeptide and the mu1 polypeptide to bind appropriately, or due to a conformational change that reduces or prohibits incorporation of the sigma3 polypeptide into the capsid.

In addition to a mutation that abolishes or reduces expression of the sigma3 polypeptide or that results in a non-functional or reduced-function sigma3 polypeptide, a mutant reovirus as described herein may contain one or more further mutations (e.g., a second, third, or fourth mutation) in one of the other reovirus capsid polypeptides (e.g., mu1, lambda2, and/or sigma1). Reoviruses containing a mutation affecting the sigma3 polypeptide and, optionally, a further mutation in any or all of the other outer capsid proteins can be screened for the ability of such mutant reoviruses to infect and cause lysis of cells. For example, neoplastic cells that are resistant to lysis by wild type reovirus can be used to screen for effective mutant reoviruses described herein.

For example, a further mutation can reduce or essentially eliminate expression of a mu1 polypeptide or result in the absence of a functional mu1 polypeptide. The mu1 polypeptide, which is encoded by the M2 gene, is likely involved in cell penetration and may play a role in transcriptase activation. Each virion contains about 600 copies of mu1 polypeptide, which are present in the form of 1:1 complexes with sigma3 polypeptides. The mu1 polypeptide is myristoylated on its N-terminus, and then the myristoylated N-terminal 42 residues are cleaved off, resulting in a C-terminal fragment (mu1C). Additionally, or alternatively, a further mutation can reduce or essentially eliminate expression of a lambda2 polypeptide or result in the absence of a functional lambda2 polypeptide, and/or a further mutation can reduce or essentially eliminate expression of a sigma1 polypeptide or result in the absence of a functional sigma1 polypeptide. The lambda2 polypeptide is encoded by the L2 gene, is involved in particle assembly, and exhibits guanylyltransferase and methyltransferase activity. The sigma1 polypeptide is encoded by the S1 gene, is involved in cell-attachment and serves as the viral hemagglutinin.

Optionally, the reovirus comprises a lambda-3 polypeptide having one or more amino acid modifications, a sigma-3 polypeptide having one or more amino acid modifications, a mu-1 polypeptide having one or more amino acid modifications, a mu-2 polypeptide having one or more amino acid modifications, or any combination thereof. For example, the reovirus has a lambda-3 polypeptide having one or more amino acid modifications; a sigma-3 polypeptide having one or more amino acid modifications; a mu-1 polypeptide having one or more amino acid modifications; and/or a mu-2 polypeptide having one or more amino acid modifications, as described in U.S. Ser. No. 12/046,095, which is incorporated by reference herein in its entirety. By way of example, the one or more amino acid modifications in the lambda-3 polypeptide are a Val at residue 214, an Ala at residue 267, a Thr at residue 557, a Lys at residue 755, a Met at residue 756, a Pro at residue 926, a Pro at residue 963, a Leu at residue 979, an Arg at residue 1045, a Val at residue 1071, or any combination thereof, numbered relative to GenBank Accession No. M24734.1 (SEQ ID NO:23). It is noted that, when the amino acid sequence is a Val at residue 214 or a Val at residue 1071, the amino acid sequence further includes at least one additional change in the amino acid sequence. Optionally, the lambda-3 polypeptide includes the sequence shown in SEQ ID NO:19. Further by way of example, the one or more amino acid modifications in the sigma-3 polypeptide are a Leu at residue 14, a Lys at residue 198, or any combination thereof, numbered relative to GenBank Accession No. K02739 (SEQ ID NO:25). It is noted that, when the amino acid sequence is a Leu at residue 14, the amino acid sequence further includes at least one additional change in the amino acid sequence. Optionally, the sigma-3 polypeptide includes the sequence shown in SEQ ID NO: 15. Further by way of example, the one or more amino acid modifications in the mu-1 polypeptide is an Asp at residue 73 numbered relative to GenBank Accession No. M20161.1 (SEQ ID NO:27). Optionally, the mu-1 polypeptide includes the sequence shown in SEQ ID NO:17. Also by way of example, the amino acid modification mu-2 polypeptide is a Ser at residue 528 numbered relative to GenBank Accession No. AF461684.1 (SEQ ID NO:29). Optionally, the mu-1 polypeptide includes the sequence shown in SEQ ID NO: 17. A reovirus as described herein having one or more modifications can further include a reovirus sigma-2 polypeptide. Such a sigma-2 polypeptide has a Cys at one or more of position 70, 127, 195, 241, 255, 294, 296, or 340, numbered relative to GenBank Accession No. NP_694684.1 (SEQ ID NO:30). Optionally, the sigma-2 polypeptide includes the sequence shown in SEQ ID NO:12.

Optionally, the reovirus comprises a L1 genome segment comprising one or more nucleic acid modifications, an S4 genome segment comprising one or more nucleic acid modifications, an M1 genome segment comprising one or more nucleic acid modifications, an M2 genome segment comprising one or more nucleic acid modifications, or any combination thereof. Optionally, the reovirus has a L1 genome segment having one or more nucleic acid modifications; a S4 genome segment having one or more nucleic acid modifications; a M1 genome segment having one or more nucleic acid modifications; and/or a M2 genome segment having one or more nucleic acid modifications, as described in WO 2008/110004, which is incorporated by reference herein in its entirety. By way of example, the one or more nucleic acid modifications in the L1 genome segment are a T at position 660, a G at position 817, an A at position 1687, a G at position 2283, an ATG at positions 2284-2286, a C at position 2794, a C at position 2905, a C at position 2953, an A at position 3153, or a G at position 3231, numbered relative to GenBank Accession No. M24734.1 (SEQ ID NO:22). Optionally, the L1 genome segment includes the sequence shown in SEQ ID NO:8. Further by way of example, the one or more nucleic acid modifications in the S4 genome segment is an A at position 74 and an A at position 624, numbered relative to GenBank Accession No. K02739 (SEQ ID NO:24). Optionally, the S4 genome segment includes the sequence shown in SEQ ID NO:4. Further by way of example, the nucleic acid modification in the M2 genome segment can be a C at position 248, numbered relative to GenBank Accession No. M20161.1 (SEQ ID NO:26). The M2 genome segment, for example, includes the sequence shown in SEQ ID NO:6. Also by way of example, the nucleic acid modification in the M1 genome segment is a T at position 1595, numbered relative to GenBank Accession No. AF461684.1 (SEQ ID NO: 28). Optionally, the M1 genome segment includes the sequence shown in SEQ ID NO:5. A reovirus as described herein can include any modification or combination of modifications disclosed herein. Optionally, a reovirus as described herein includes genomic segments having the sequences shown in SEQ ID NOs: 1-10 or the polypeptides shown in SEQ ID NOs: 11, 12, and 16-21, and either or both of the sequences shown in SEQ ID NO: 13 and 14. Optionally, a reovirus as disclosed herein is identified as IDAC Accession No. 190907-01, which was deposited with the International Depositary of Canada (IDAC, National Microbiology Laboratory, Public Health Agency of Canada, 1015 Arlington St., Winnipeg, Manitoba Canada R3E 3R2 on Sep. 19, 2007.

Sindbis virus (SIN) can be used in the methods described herein. Sindbis virus is a member of the alphavirus genus of the togaviridae family. The Sindbis virus genome is a single-stranded RNA of 11703 nucleotides, capped at the 5′ terminus and poly-adenylated at the 3′ terminus. The genome consists of a 49S untranslated region (UT), nonstructural proteins nsP1, nsP2, nsP3, and nsP4 followed by a promoter. The promoter is followed by a 26S UT, structural proteins C, E3, E2, 6K, and E1 and finally a 3′ UT and a poly-adenylated terminus. The genomic 49S RNA is of plus sense, is infectious, and serves as mRNA in the infected cell.

Sindbis vectors systemically and specifically infect/detect and kill metastasized tumors in vivo, leading to significant suppression of tumor growth and enhanced survival (Hurtado et al., Rejuvenation Res. 9(1): 36-44 (2006)). Sindbis virus infects mammalian cells using the Mr 67,000 laminin receptor, which is elevated in tumor versus normal cells. Tumor overexpression of the laminin receptor may explain the specificity and efficacy that Sindbis vectors demonstrate for tumor cells in vivo. Sindbis does not have to undergo genetic manipulation to target cancer cells or to be injected directly into tumors. Sindbis injected anywhere into a subject travels through the bloodstream to the target area (Tseng et al., Cancer Res. 64(18): 6684-92 (2004). Sindbis can also be genetically engineered to carry one or more genes that suppress the immune response to the virus and/or genes that stimulate an immune response against the tumor such as, for example, antitumor cytokine genes such as interleukin-12 and interleukin-15 genes.

The virus may be chemically or biochemically pretreated (e.g., by treatment with a protease, such as chymotrypsin or trypsin) prior to administration to the neoplastic cells. Pretreatment with a protease removes the outer coat or capsid of the virus and may increase the infectivity of the virus. The virus may be coated in a liposome or micelle (Chandran and Nibert, J. of Virology 72(1): 467-75 (1998)) to reduce or prevent an immune response from a mammal which has developed immunity to the virus. For example, the virion may be treated with chymotrypsin in the presence of micelle forming concentrations of alkyl sulfate detergents to generate a new infectious subvirion particle. The oncolytic virus may also be a reassortant virus or an ISVP.

The oncolytic virus may be a recombinant oncolytic virus. For example, the recombinant oncolytic virus results from the reassortment of genomic segments from two or more genetically distinct oncolytic viruses, also referred to herein as a reassortant. Reassortment of oncolytic virus genomic segments may occur following infection of a host organism with at least two genetically distinct oncolytic viruses. Recombinant viruses can also be generated in cell culture, for example, by co-infection of permissive host cells with genetically distinct oncolytic viruses. Optionally, the methods include the use of recombinant oncolytic virus resulting from reassortment of genome segments from two or more genetically distinct oncolytic viruses wherein at least one parental virus is genetically engineered, comprises one or more chemically synthesized genomic segment, has been treated with chemical or physical mutagens, or is itself the result of a recombination event. Optionally, the methods include the use of the recombinant oncolytic virus that has undergone recombination in the presence of chemical mutagens, including but not limited to dimethyl sulfate and ethidium bromide, or physical mutagens, including but not limited to ultraviolet light and other forms of radiation.

Optionally, the methods include the use of oncolytic viruses with mutations including (insertions, substitutions, deletions or duplications) in one or more genome segments. Such mutations can comprise additional genetic information as a result of recombination with a host cell genome, or that comprise synthetic genes such as, for example, genes encoding agents that suppress anti-viral immune responses.

Optionally, the oncolytic virus is a mutant oncolytic virus. For example, the oncolytic virus may be modified by incorporation of mutated coat proteins, such as for example, into the virion outer capsid. The mutant oncolytic virus is, optionally, a mutant reovirus. Mutant reoviruses as described herein can contain a mutation that reduces or essentially eliminates expression of a sigma3 polypeptide or that results in the absence of a functional sigma3 polypeptide as described in U.S. Publication No. 2008/0292594, which is incorporated by reference herein in its entirety. Optionally, the mutant reoviruses used in the provided methods are mutated as described in U.S. Pat. No. 7,803,385, which is incorporated by reference herein in its entirety.

A mutation as referred to herein can be a substitution, insertion or deletion of one or more nucleotides. Point mutations include, for example, single nucleotide transitions (purine to purine or pyrimidine to pyrimidine) or transversions (purine to pyrimidine or vice versa) and single- or multiple-nucleotide deletions or insertions. A mutation in a nucleic acid can result in one or more conservative or non-conservative amino acid substitutions in the encoded polypeptide, which may result in conformational changes or loss or partial loss of function, a shift in the reading frame of translation (frame-shift) resulting in an entirely different polypeptide encoded from that point on, a premature stop codon resulting in a truncated polypeptide (truncation), or a mutation in a virus nucleic acid may not change the encoded polypeptide at all (silent or nonsense). See, for example, Johnson and Overington, 1993, J. Mol. Biol. 233:716-38; Henikoff and Henikoff, 1992, Proc. Natl. Acad. Sci. USA 89:10915-19; and U.S. Pat. No. 4,554,101, for disclosure on conservative and non-conservative amino acid substitutions.

Mutations can be generated in the nucleic acid of an oncolytic virus using any number of methods known in the art. For example, site directed mutagenesis can be used to modify a reovirus nucleic acid sequence. One of the most common methods of site-directed mutagenesis is oligonucleotide-directed mutagenesis. In oligonucleotide-directed mutagenesis, an oligonucleotide encoding the desired change(s) in sequence is annealed to one strand of the DNA of interest and serves as a primer for initiation of DNA synthesis. In this manner, the oligonucleotide containing the sequence change is incorporated into the newly synthesized strand. See, for example, Kunkel, 1985, Proc. Natl. Acad. Sci. USA 82:488; Kunkel et al., 1987, Meth. Enzymol. 154:367; Lewis and Thompson, 1990, Nucl. Acids Res. 18:3439; Bohnsack, 1996, Meth. Mol. Biol. 57:1; Deng and Nickoloff, 1992, Anal. Biochem. 200:81; and Shimada, 1996, Meth. Mol. Biol. 57:157. Other methods are used routinely in the art to modify the sequence of a protein or polypeptide. For example, nucleic acids containing a mutation can be generated using PCR or chemical synthesis, or polypeptides having the desired change in amino acid sequence can be chemically synthesized. See, for example, Bang and Kent, 2005, Proc. Natl. Acad. Sci. USA 102:5014-9 and references therein.

Also provided are compositions comprising the virus collected according to the methods provided herein and such compositions can include, for example, a pharmaceutically acceptable excipient. The herein provided compositions are administered in vitro or in vivo in a pharmaceutically acceptable carrier. A pharmaceutically acceptable carrier can be a solid, semi-solid, or liquid material that can act as a vehicle, carrier or medium for the reovirus. Thus, compositions containing an oncolytic virus and/or one or more of the provided agents can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Optionally, the compositions containing an oncolytic virus are suitable for infusion. For intravenous infusions, there are two types of fluids that are commonly used, crystalloids and colloids. Crystalloids are aqueous solutions of mineral salts or other water-soluble molecules. Colloids contain larger insoluble molecules, such as gelatin; blood itself is a colloid. The most commonly used crystalloid fluid is normal saline, a solution of sodium chloride at 0.9% concentration, which is close to the concentration in the blood (isotonic). Ringer's lactate or Ringer's acetate is another isotonic solution often used for large-volume fluid replacement. A solution of 5% dextrose in water, sometimes called D5W, is often used instead if the patient is at risk for having low blood sugar or high sodium.

Some examples of suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. A pharmaceutical composition additionally can include, without limitation, lubricating agents such as talc, magnesium stearate, and mineral oil; wetting agents; emulsifying and suspending agents; preserving agents such as methyl- and propylhydroxy-benzoates; sweetening agents; and flavoring agents. Pharmaceutical compositions can be formulated to provide quick, sustained or delayed release of a mutant reovirus after administration by employing procedures known in the art. In addition to the representative formulations described below, other suitable formulations for use in a pharmaceutical composition can be found in Remington: The Science and Practice of Pharmacy 22d edition Loyd V. Allen et al, editors, Pharmaceutical Press (2012). For preparing solid compositions such as tablets, a mutant reovirus can be mixed with a pharmaceutical carrier to form a solid composition. Optionally, tablets or pills can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid formulations that include a reovirus and/or agent for oral administration or for injection generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. These liquid or solid compositions may contain suitable pharmaceutically acceptable excipients as described herein. Such compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent or intermittent positive pressure breathing machine. Solution, suspension, or powder compositions may be administered, orally or nasally, from devices which deliver the formulation in an appropriate manner.

Another formulation that is optionally employed in the methods of the present disclosure includes transdermal delivery devices (e.g., patches). Such transdermal patches may be used to provide continuous or discontinuous infusion of the viruses and agents as described herein. The construction and use of transdermal patches for the delivery of pharmaceutical agents is well known in the art. See, for example, U.S. Pat. No. 5,023,252. Such patches can be constructed for continuous, pulsatile, or on-demand delivery of mutant reoviruses.

As described above, viruses and/or other agents can, if necessary, be coated in a liposome or micelle to reduce or prevent an immune response in a mammal that has developed immunity toward a virus or agent. Such compositions are referred to as immunoprotected viruses or agents. See, for example, U.S. Pat. Nos. 6,565,831 and 7,014,847.

In the provided methods, the oncolytic virus is administered, for example, systemically, in a manner so that it can ultimately contact the target tumor or tumor cells. The route by which the virus is administered, as well as the formulation, carrier or vehicle, depends on the location as well as the type of the target cells. A wide variety of administration routes can be employed. For example, for a solid tumor that is accessible, the virus can be administered by injection directly to the tumor. For a hematopoietic tumor, for example, the virus can be administered intravenously or intravascularly. For tumors that are not easily accessible within the body, such as metastases, the virus is administered in a manner such that it can be transported systemically through the body of the mammal and thereby reach the tumor (e.g., intravenously or intramuscularly). Alternatively, the virus can be administered directly to a single solid tumor, where it then is carried systemically through the body to metastases. The virus can also be administered subcutaneously, intraperitoneally, intrathecally or intraventricularly (e.g., for brain tumors), topically (e.g., for melanomas), orally (e.g., for oral or esophageal cancers), rectally (e.g., for colorectal cancers), vaginally (e.g., for cervical or vaginal cancers), nasally, by inhalation spray or by aerosol formulation (e.g., for lung cancers).

Optionally, the virus is administered continuously to a subject at least once per day or up to intermittently or continuously throughout the day on consecutive days, for a period of time for a first or subsequent round of treatment. Thus, the virus is administered, for example, to subjects by means of intravenous administration in any pharmacologically acceptable solution, or as an infusion over a period of time. For example, the substance may be administered systemically by injection (e.g., IM or subcutaneously) or taken orally daily at least once per day, or administered by infusion in a manner that results in the daily delivery into the tissue or blood stream of the subject. When the virus is administered by infusion over a period of time, the period of time is, for example, 1, 2, 3, 4, 5, 6, 7, 8, 9, 10, 12, or 24 hours, or any time between 1 and 24 hours, inclusive, or more. Optionally, the period of time is 5, 15, 30, 60, 90, 120, 150 or 180 minutes, or any time between 5 and 180 minutes, inclusive, or more. Thus, for example, the virus is administered by infusion for 60 minutes. Administrations can be repeated daily for 2, 3, 4, 5, 6, 7, 8, 9, 10, 14, 21, 28 days or any number of days between 2 and 28 days, inclusive, or longer.

The viruses as disclosed herein are administered in an amount that is sufficient (i.e., an effective amount) to effect the treatment of the cancer or proliferative disorder. A cancer or proliferative disorder is treated when administration of a treatment regimen including a virus to proliferating cells affects lysis (e.g., oncolysis) of the affected cells, resulting in a reduction in the number of abnormally, proliferating cells, a reduction in the size of a neoplasm, and/or a reduction in or elimination of symptoms (e.g., pain) associated with the proliferating disorder. As used herein, the term oncolysis means at least 10% of the proliferating cells are lysed (e.g., at least about 20%, at least about 30%, at least about 40%, at least about 50%, or at least about 75% of the cells are lysed). The percentage of lysis can be determined, for example, by measuring the reduction in the size of a neoplasm or in the number of proliferating cells in a mammal, or by measuring the amount of lysis of cells in vitro (e.g., from a biopsy of the proliferating cells). An effective amount of a virus used in a treatment regimen will be determined on an individual basis and may be based, at least in part, on the particular virus used; the individual's size, age, gender; and the size and other characteristics of the abnormally, proliferating cells. For example, for treatment of a human, approximately 10³ to 10¹² plaque forming units (PFU) of a virus are used, depending on the type, size and number of proliferating cells or neoplasms present. The effective amount can be, for example, from about 1.0 PFU/kg body weight to about 10¹⁵ PFU/kg body weight (e.g., from about 10² PFU/kg body weight to about 10¹³ PFU/kg body weight). Optionally, the effective amount is about 1×10⁸ to about 1×10¹² PFU or TCID50. Optionally, the effective amount is about 3×10¹⁰ to about 1×10¹⁰ TCID50. By way of example, the effective amount can be between 3.0×10¹⁰ to 4.5×10¹⁰ TCID50. Optionally, the effective amount is 4.5×10¹⁰ TCID50. Optionally, vials containing the viruses can include, for example, 5×10⁹ to 1×10¹¹ TCID50/mL.

Optimal dosages of viruses and therapeutic agents, and compositions and kits comprising viruses and agents depend on a variety of factors. The exact amount required will vary from subject to subject, depending on the species, age, weight and general condition of the subject, the severity of the disease being treated, the particular virus and its mode of administration. Thus, it is not possible to specify an exact amount for every composition or kit. However, an appropriate amount can be determined by one of ordinary skill in the art using only routine experimentation given the guidance provided herein.

Effective dosages and schedules for administering the treatment regimens may be determined empirically. For example, animal models for a variety of proliferative disorders can be obtained from the Jackson Laboratory, 600 Main Street, Bar Harbor, Maine 04609 USA. Both direct (e.g., histology of tumors) and functional measurements (e.g., survival of a subject or size of a tumor) can be used to monitor response to therapies. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy. The dosage ranges for the administration of compositions are those large enough to produce the desired effect in which the symptoms of the disease are affected. The dosage should not be so large as to cause adverse side effects, such as unwanted cross-reactions and anaphylactic reactions. The dosage can be adjusted by the individual physician in the event of any counterindications.

Dosages vary and are administered in one or more dose administrations, for example, daily, for one or several days. The provided viruses and therapeutic agents are administered in a single dose or in multiple doses (e.g., two, three, four, six, or more doses). For example, where the administration is by infusion, the infusion can be a single sustained dose or can be delivered by multiple infusions. Treatment may last from several days to several months or until diminution of the disease is achieved.

The provided methods may be further combined with other tumor therapies such as chemotherapy, radiotherapy, surgery, hormone therapy and/or other immunotherapies. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

Combinations of the provided viruses and therapeutic agents are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

When one compound is administered prior to another compound, the first compound is administered minutes, hours, days, or weeks prior to administration of the second compound. For example, the first compound can be administered at 1, 2, 3, 4, 5, 6, 7, 8, 9, 10, 12, 24, 36, 48, 60, or 72 hours, or any time between 1 and 72 hours, inclusive, prior to administration of a second compound. Optionally, the first compound is administered more than 72 hours prior to the second compound. By way of another example, the first compound can be administered at 1, 5, 15, 30, 60, 90, 120, 150 or 180 minutes, or any time between 1 and 180 minutes, inclusive, prior to administration of a second compound. Optionally, the first compound is administered at 1, 2, 3, 4, 5, 6, 7, 14, 21, or 28 days, or at any timepoint between 1 and 28, inclusive, days prior to administration of the second compound. Optionally, the first compound is administered more than 28 days prior to the second compound.

Oncolytic viruses or a pharmaceutical composition comprising such viruses can be packaged into a kit. The kit also includes one or more additional agents or pharmaceutical compositions comprising the additional agents. The kit can include chemotherapeutic agents or cancer immunotherapeutic agents. Optionally, the kit includes an immune checkpoint inhibitor.

The oncolytic viruses and/or additional agents and pharmaceutical compositions containing the same can be packaged in one or more containers. When the kits contain pharmaceutical compositions, the pharmaceutical compositions can be formulated in a unit dosage form. The term “unit dosage forms” refers to physically discrete units suitable as unitary dosages for human subjects and other mammals, each unit containing a predetermined quantity of an oncolytic virus or other agent, e.g., immune checkpoint inhibitor calculated to produce the desired therapeutic effect in association with a suitable pharmaceutically acceptable carrier. Optionally, the kit includes a reovirus and an immune checkpoint inhibitor.

The oncolytic virus in the provided kits can be any of the oncolytic viruses described herein. The provided kits can include more than one dose of the oncolytic virus. Optionally, each dose of oncolytic virus comprises approximately 10³ to 10¹² plaque forming units (PFU) of the oncolytic virus. Optionally, each dose comprises approximately 10⁸ to 10¹² PFU of the oncolytic virus. Optionally, each dose comprises approximately 10⁸ to 10¹² TCID50 of the oncolytic virus. Optionally, each dose comprises approximately 1×10¹⁰ to 3×10¹⁰ TCID50 of the oncolytic virus. By way of example, each dose can be between 3.0×10¹⁰ to 4.5×10¹⁰ TCID50. Optionally, each dose is 4.5×10¹⁰ TCID50. Optionally, vials containing the viruses can include, for example, 5×10⁹ to 1×10¹¹ TCID50/mL.

As used herein the terms treatment, treat, treating or ameliorating refers to a method of reducing the effects of a disease or condition or symptom of the disease or condition. Thus, in the disclosed method, treatment can refer to a 10% or greater, 20% or greater, 30% or greater, 40% or greater, 50% or greater, 60% or greater, 70% or greater, 80% or greater, 90% or greater or 100% reduction or amelioration in the severity of an established disease or condition or symptom of the disease or condition. For example, the method for treating cancer is considered to be a treatment if there is a 10% or greater reduction in one or more symptoms of the disease in a subject as compared to control. Thus, the reduction can be 10% or greater, 20% or greater, 30% or greater, 40% or greater, 50% or greater, 60% or greater, 70% or greater, 80% or greater, 90% or greater, 100% or any percent reduction in the range of 10% to 100% as compared to native or control levels. It is understood that treatment does not necessarily refer to a cure or complete ablation of the disease, condition or symptoms of the disease or condition.

As used herein, the term subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, whether male or female, are intended to be covered. As used herein, the terms patient and subject may be used interchangeably and can refer to a subject with a disease or disorder. The term patient or subject includes human and veterinary subjects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of the inhibitor, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described, it can be combined with any other characteristic or step herein even if the combination is not explicitly stated. Accordingly, other aspects are within the scope of the claims.

### EXAMPLES

### Example 1. Lysis, Extraction and Purification of Reovirus from Host Cells

1% Triton X-100 is used in the current validated reovirus manufacturing process to lyse HEK293 cells, to maximize viral particle recovery at the upstream harvest step. However, Triton X-100 was added to the REACH list and cannot be used for commercial production in the European Union due to its environmental impact. Therefore, an alternative detergent needs to be identified and tested to avoid any delays in reovirus manufacture.

Table 1 is a list of alternatives tested in this example.

TABLE 1
List of Detergents.

| | | Grade Available/ | REACH |
|---|---|---|---|
| Detergent | Type | Quality Level | Status |
| Deviron ® 16 | Zwitterionic | Quality Level 400 | Low risk |
| ECOSURF ™-EH9 | Non-ionic | Quality Level 200 | Low risk |
| Octyl β-D-Glucopyranoside (OGP) | Non-ionic | Quality Level 300 | Low risk |
| Sodium Deoxycholate (NaDOC) | Anionic | Quality Level 400 | Low risk |
| Tween 20 | Non-ionic | USP, Multi-compendial | Low risk |
| Tween 80 | Non-ionic | USP, Multi-compendial | Low risk |
| Tergitol 15-S-9 | Non-ionic | Quality Level 200 | Low risk |

Alternative candidate detergents were assessed at varying concentrations (0.5%, 1%, 2%) and durations (1 hour, 2 hours, 3 hours) during the lysis step, followed by incubation with Benzonase to digest host cell DNA (HC DNA). The detergents tested were Deviron C16; ECOSURF EH-9; Octyl β-D-Glucopyranoside (OGP); Sodium Deoxycholate (NaDOC); Tween 20; Tween 80, and Tergitol 15-S-9. Samples were analyzed for virus particle recovery (HPLC), infectious viral particles (TCID50), and residual HC DNA. These data were compared to Triton X-100 and cells lysed via freeze/thaw cycles as controls. The results are shown in Tables 2 and 3.

TABLE 2
Alternative Detergents - Viral Titer Results (HPLC)

| | | Detergent |
|---|---|---|
| | HPLC Titer | Efficiency vs |
| Conditions | (vp/mL) | Triton X-100 (%) |
| Supernatant CTL - centrifuged 3 dpi cell culture | 4.66 × 10¹¹ | |
| Triton X-100 0.91% CTL, 1 hr incubation | 4.67 × 10¹¹ | 100 |
| ECOSURF- ES-9 0.5%, 1 hr incubation | 3.02 × 10¹¹ | 64.7 |
| ECOSURF- ES-9 1.0%, 1 hr incubation | 3.09 × 10¹¹ | 66.2 |
| ECOSURF- ES-9 2.0%, 1 hr incubation | 2.14 × 10¹¹ | 45.8 |
| ECOSURF- ES-9 0.5%, 2 hr incubation | No reovirus peak | 0 |
| ECOSURF- ES-9 1.0%, 2 hr incubation | No reovirus peak | 0 |
| ECOSURF- ES-9 2.0%, 2 hr incubation | No reovirus peak | 0 |
| ECOSURF- ES-9 0.5%, 3 hr incubation | No reovirus peak | 0 |
| ECOSURF- ES-9 1.0%, 3 hr incubation | No reovirus peak | 0 |
| ECOSURF- ES-9 2.0%, 3 hr incubation | No reovirus peak | 0 |
| Tergitol 15-S-9 0.5%, 1 hr incubation | 3.12 × 10¹¹ | 66.8 |
| Tergitol 15-S-9 1.0%, 1 hr incubation | 2.48 × 10¹¹ | 53.1 |
| Tergitol 15-S-9 2.0%, 1 hr incubation | 3.15 × 10¹¹ | 67.5 |
| Tergitol 15-S-9 0.5%, 2 hr incubation | 4.19 × 10¹¹ | 89.7 |
| Tergitol 15-S-9 1.0%, 2 hr incubation | 4.55 × 10¹¹ | 97.4 |
| Tergitol 15-S-9 2.0%, 2 hr incubation | 4.33 × 10¹¹ | 92.7 |
| Tergitol 15-S-9 0.5%, 3 hr incubation | 3.59 × 10¹¹ | 76.9 |
| Tergitol 15-S-9 1.0%, 3 hr incubation | 3.61 × 10¹¹ | 77.3 |
| Tergitol 15-S-9 2.0%, 3 hr incubation | 3.90 × 10¹¹ | 83.5 |

dpi: days post infection;
CTL: control;
hr: hour;
vp: viral particle

TABLE 3
Alternative Detergents - Viral Titer Results (HPLC)

| | | Detergent |
|---|---|---|
| | HPLC Titer | Efficiency vs |
| Conditions | (vp/mL) | Triton X-100 (%) |
| Supernatant CTL - centrifuged 3 dpi cell culture | 3.18 × 10¹¹ | |
| Triton X-100 0.91% CTL, 1 hr incubation | 3.62 × 10¹¹ | 100 |
| Octyl ß-D-Glucopyranoside 0.5%, 1 hr incubation | 3.40 × 10¹¹ | 93.9 |
| Octyl ß-D-Glucopyranoside 1.0%, 1 hr incubation | 2.82 × 10¹¹ | 77.9 |
| Octyl ß-D-Glucopyranoside 1.5%, 1 hr incubation | 1.86 × 10¹¹ | 51.4 |
| Deviron C16 0.1%, 1 hr incubation | 2.67 × 10¹¹ | 73.8 |
| Deviron C16 0.25%, 1 hr incubation | 2.02 × 10¹¹ | 55.8 |
| Deviron C16 0.5%, 1 hr incubation | 9.20 × 10¹⁰ | 25.4 |
| Sodium deoxycholate 0.1%, 1 hr incubation | 4.19 × 10¹¹ | 115.8 |
| Sodium deoxycholate 0.25%, 1 hr incubation | 3.12 × 10¹¹ | 86.2 |
| Sodium deoxycholate 0.5%, 1 hr incubation | No reovirus peak | N/A |

dpi: days post infection;
CTL: control;
hr: hour;
vp: viral particle

Conditions resulting in viral particle recoveries >80% versus the Triton X-100 control were selected for full downstream analysis. Viral recoveries ranging from 90-98% resulted from 0.5% Octyl β-D-Glucopyranoside (OGP) and 2 hr Tergitol 15-S-9 (at 0.5%, 1%, 2%) conditions. Sodium Deoxycholate (NaDOC) at 0.1% showed the best efficiency, 16% higher than the Triton X-100. However, there is a trend where increasing NaDOC concentrations results in decreasing viral titer with no detectable viral particles for the 0.5% NaDOC concentration. Also, because NaDOC is an anionic detergent, it is not compatible with the ion exchange purification step, unless it is first removed from the sample before purification using the ion exchange column. Therefore, it was decided not to continue with this detergent. As noted for NaDOC above, OGP and Deviron C-16 also show decreasing titer with increasing detergent concentrations, suggesting these higher detergent amounts may degrade reovirus.

Tween 20 was tested at varying concentrations (0.5%, 1%, 1.5%) and incubation times (1 hr, 2 hr, 3 hr). The results are shown in Table 4.

TABLE 4
Tween 20 Viral Titer Results (HPLC)

| | | Detergent |
|---|---|---|
| | HPLC Titer | Efficiency vs |
| Conditions | (vp/mL) | Triton X-100 (%) |
| Supernatant CTL - centrifuged 3 dpi cell culture | 4.66 × 10¹¹ | |
| Triton X-100 0.91% CTL, 1 hr incubation | 4.67 × 10¹¹ | 100 |
| Tween 20 0.5%, 1 hr incubation | 2.77 × 10¹¹ | 59.3 |
| Tween 20 1.0%, 1 hr incubation | 2.82 × 10¹¹ | 60.4 |
| Tween 20 1.5%, 1 hr incubation | 3.04 × 10¹¹ | 65.1 |
| Tween 20 0.5%, 2 hr incubation | 3.03 × 10¹¹ | 64.9 |
| Tween 20 1.0%, 2 hr incubation | 2.76 × 10¹¹ | 59.1 |
| Tween 20 1.5%, 2 hr incubation | 2.74 × 10¹¹ | 58.7 |
| Tween 20 0.5%, 3 hr incubation | 2.87 × 10¹¹ | 61.4 |
| Tween 20 1.0%, 3 hr incubation | 2.81 × 10¹¹ | 60.2 |
| Tween 20 1.5%, 3 hr incubation | 2.78 × 10¹¹ | 59.5 |

dpi: days post infection;
CTL: control;
hr: hour;
vp: viral particle

None of these conditions resulted in viral particle recoveries >80% versus Triton X-100, therefore, further optimization of the Tween 20 lysis conditions was required. These results are shown in Table 5.

TABLE 5
Tween 20 Viral Titer Results (HPLC).

| | | Detergent |
|---|---|---|
| | HPLC Titer | Efficiency vs |
| Conditions | (vp/mL) | Triton X-100 (%) |
| Supernatant CTL - centrifuged 3 dpi cell culture | 3.18 × 10¹¹ | |
| Triton X-100 0.91% CTL | 3.62 × 10¹¹ | 100 |
| Tween 20 0.5% - Tris 50 mM pH 7, 1 hr incubation | 2.71 × 10¹¹ | 74.9 |
| Tween 20 0.5% - Tris 50 mM pH 7.5, 1 hr incubation | 2.68 × 10¹¹ | 74.0 |
| Tween 20 0.5% - Tris 50 mM pH 8, 1 hr incubation | 2.74 × 10¹¹ | 75.7 |
| Tween 20 0.5% - PO4 10 mM pH 7, 1 hr incubation | 2.62 × 10¹¹ | 72.4 |
| Tween 20 0.5% - PO4 10 mM pH 7.5, 1 hr incubation | 2.58 × 10¹¹ | 71.3 |
| Tween 20 0.5% - PO4 10 mM pH 8, 1 hr incubation | 2.57 × 10¹¹ | 71.0 |
| Tween 20 0.5% - PO4 20 mM pH 7, 1 hr incubation | 2.62 × 10¹¹ | 72.4 |
| Tween 20 0.5% - PO4 20 mM pH 7.5, 1 hr incubation | 2.71 × 10¹¹ | 74.9 |
| Tween 20 0.5% - PO4 20 mM pH 8, 1 hr incubation | 2.72 × 10¹¹ | 75.1 |
| Tween 20 1% - Tris 50 mM pH 7, 1 hr incubation | 2.47 × 10¹¹ | 68.2 |
| Tween 20 1% - Tris 50 mM pH 7.5, 1 hr incubation | 2.40 × 10¹¹ | 66.3 |
| Tween 20 1% - Tris 50 mM pH 8, 1 hr incubation | 2.43 × 10¹¹ | 67.1 |
| Tween 20 1% - PO4 10 mM pH 7, 1 hr incubation | 2.36 × 10¹¹ | 65.2 |
| Tween 20 1% - PO4 10 mM pH 7.5, 1 hr incubation | 2.42 × 10¹¹ | 66.9 |
| Tween 20 1% - PO4 10 mM pH 8, 1 hr incubation | 2.38 × 10¹¹ | 65.7 |
| Tween 20 1% - PO4 20 mM pH 7, 1 hr incubation | 2.40 × 10¹¹ | 66.3 |
| Tween 20 1% - PO4 20 mM pH 7.5, 1 hr incubation | 2.76 × 10¹¹ | 76.2 |
| Tween 20 1% - PO4 20 mM pH 8, 1 hr incubation | 2.94 × 10¹¹ | 81.2 |

dpi: days post infection;
CTL: control;
hr: hour;
PO4: Sodium Phosphate;
vp: viral particle

There is a positive trend for the 20 mM sodium phosphate (PO4) buffer lysis condition with increasing pH. The 1% Tween 20 results are better than the 0.5% Tween 20 results for the same pH conditions. The 1% Tween 20 with 20 mM sodium phosphate at pH 8 shows a titer above the cut-off value of 80% of Triton X-100. However, the Tween 20 lysis conditions vs Triton X-100 were not improved using 10 mM sodium phosphate buffer, or 50 mM Tris buffer at pH 7.0, 7.5, or 8.0.

The results of further optimization of the 1% Tween 20 lysis conditions are shown in Table 6.

TABLE 6
Further Optimization of Tween 20 - Viral Titer Results (HPLC).

| | | Detergent |
|---|---|---|
| | HPLC Titer | Efficiency vs |
| Conditions | (vp/mL) | Triton X-100 (%) |
| Supernatant CTL - centrifuged 3 dpi cell culture | 3.18 × 10¹¹ | |
| Triton X-100 0.91% CTL | 3.28 × 10¹¹ | 100 |
| Tween20 1%, 2 hr, salt −80° C. | 3.32 × 10¹¹ | 101 |
| Tween20 1%, 2 hr, salt O/N 4° C.-80° C. | 2.92 × 10¹¹ | 88 |
| Tween20 1%, 2 hr −80° C. | 3.24 × 10¹¹ | 99 |
| Tween20 1%, 2 hr O/N 4° C.-80° C. | 2.77 × 10¹¹ | 83 |
| Tween20 1%, 3 hr, salt −80° C. | 2.85 × 10¹¹ | 87 |
| Tween20 1%, 3 hr, salt O/N 4° C.-80° C. | 2.81 × 10¹¹ | 85 |
| Tween20 1%, 3 hr −80° C. | 3.21 × 10¹¹ | 98 |
| Tween20 1%, 3 hr O/N 4° C.-80° C. | 2.86 × 10¹¹ | 86 |
| Tween20 1%, 4 hr, salt −80° C. | 2.84 × 10¹¹ | 87 |
| Tween20 1%, 4 hr, salt O/N 4° C.-80° C. | 2.85 × 10¹¹ | 86 |
| Tween80 1%, 1 hr −80° C. | 3.03 × 10¹¹ | 92 |
| Tween80 1%, 1 hr O/N 4° C.-80° C. | 2.79 × 10¹¹ | 84 |
| Tween80 1%, 2 hr, salt −80° C. | 3.12 × 10¹¹ | 95 |
| Tween80 1%, 2 hr, salt O/N 4° C.-80° C. | 2.59 × 10¹¹ | 78 |
| Tween80 1.5%, 1 hr −80° C. | 2.84 × 10¹¹ | 87 |
| Tween80 1.5%, 1 hr O/N 4° C.-80° C. | 2.77 × 10¹¹ | 83 |
| Tween80 1.5%, 2 hr, salt −80° C. | 3.09 × 10¹¹ | 94 |
| Tween80 1.5%, 2 hr, salt O/N 4° C.-80° C. | 2.82 × 10¹¹ | 85 |

dpi: days post infection;
CTL: control;
hr: hour;
O/N: overnight;
vp: viral particle

Salt in Table 6 refers to 200 mM NaCl, which was included to see if it would improve the Tween 20 lysis conditions.

Further optimization of the 1% Tween 20, 20 mM phosphate lysis buffer, pH 8 included longer detergent incubation times (1 hr, 2 hr, 3 hr) and the addition of 200 mM NaCl post-Benzonase digestion. For both Tween detergents (Tween 20 and Tween 80), the addition of 200 mM NaCl did not improve viral particle recovery. Incubation for 2 hours with Tween 20 resulted in the highest HPLC recoveries. Lysis with Tween 80 resulted in HPLC recoveries >80% vs. Triton X-100. However, as the Tween 80 results are comparable to Tween 20, it was decided to move forward with Tween 20.

Further analysis of alternative detergents for the replacement of Triton X-100, including infectious viral titers, are shown in Table 7. Note that although the Triton X-100 control appears to have higher HPLC and TCID50 recoveries than Tween 20, at the larger manufacturing scale (1.25 L), this trend is reversed, with Tween 20 resulting in improved HPLC and TCID50 titers versus Triton X-100.

TABLE 7
Triton X-100 Replacement-Detergent Viral Titer (TCID50) Results

| | | Detergent |
|---|---|---|
| | TCID50 Titer | Efficiency vs |
| Conditions | (TCID50/mL) | Triton X-100 (%) |
| Supernatant CTL - centrifuged 3 dpi cell culture | 8.46 × 10⁰⁹ | |
| Triton X-100 0.91% CTL | 1.63 × 10¹⁰ | 100 |
| Tween 20 1% - PO4 20 mM pH 7 | 9.81 × 10⁰⁹ | 60.1 |
| Tween 20 1% - PO4 20 mM pH 7.5 | 9.76 × 10⁰⁹ | 59.8 |
| Tween 20 1% - PO4 20 mM pH 8 | 1.48 × 10¹⁰ | 90.7 |
| Octyl ß-D-Glucopyranoside 0.5% | 1.64 × 10¹⁰ | 100.3 |
| Octyl ß-D-Glucopyranoside 1.0% | 7.68 × 10⁰⁹ | 47.1 |
| Octyl ß-D-Glucopyranoside 1.0% 4° C. O/N | 3.00 × 10¹⁰ | Invalid assay result |
| Deviron C16 0.1% | 4.27 × 10⁰⁹ | 26.2 |
| Deviron C16 0.25% | 5.00 × 10⁰⁸ | 3.1 |
| Deviron C16 0.25% 4° C. O/N | 1.30 × 10⁰⁹ | 8.0 |
| Sodium deoxycholate 0.1% | 2.10 × 10¹⁰ | 128.9 |
| Sodium deoxycholate 0.25% | 1.58 × 10¹⁰ | 96.7 |
| Sodium deoxycholate 0.25% 4° C. O/N | 1.76 × 10¹⁰ | 107.6 |

dpi: days post infection;
CTL: control;
PO4: Sodium Phosphate;
O/N: overnight incubation at 4° C.

The TCID50 viral titer results show a similar trend to the HPLC data. Detergents resulting in the best recovery of infectious viral titer versus Triton X-100 were 1% Tergitol 15-S-9, 1% Tween 20 in 20 mM PO4 buffer pH 8, 0.5% OGP, and 0.1% NaDOC.

In summary, the screening of different alternative detergents identified a few potential candidates (OGP, NaDOC, Tween 20, Tergitol 15-S-9) to replace the Triton X-100.

Three conditions were used to assess the detergents' impact on the manufacturing process at the 1.25 L scale versus the current Triton X-100 process (Table 8). The following detergents and conditions were tested: 1% Tween 20 with 20 mM NaPO₄ lysis buffer pH 8.0, 2 hr incubation; 1% Tergitol 15-S-9 for 1 hr incubation; and 0.5% OGP for 1 hr incubation. For Tergitol 15-S-9, the total HPLC recovery was 21% greater than Triton X-100 (5.28×10¹³ vs 4.35×10¹³), and the total infectious viral particles (TCID50) was 25% higher than Triton X-100 (3.22×10¹² vs 2.57×10¹²). For 0.5% OGP, the total viral particle yield was 8.24×10¹³ HPLC and total infectious viral particle yield was 2.10×10¹² TCID50. For both total viral particle yield (HPLC) and infectious viral particle yield (TCID50), the Tween 20 results are better than the Triton X-100. Tween 20 resulted in 54% and 29% higher yield than Triton X-100 for total HPLC (1.53×10¹⁴ HPLC vs 9.94×10¹³ HPLC) and TCID50 (3.27×10¹² vs 2.53×10¹²), respectively. These are the best yields of the 3 detergents tested in the reovirus production method at the 1.25 L scale.

TABLE 8
Summary of Bulk Purified Material Using 3 Different Detergents (1.25 L Manufacturing Scale)

| | Viral | | Infectious Viral | | Host Cell | | Host Cell | |
|---|---|---|---|---|---|---|---|---|
| | Particles | | Particles | | Protein | | DNA (HC | |
| | (HPLC) | | (TCID50) | | (HCP) | | DNA) | |
| Detergent | vp/ | Total | TCID50/ | Total | ng/ | | ng/ | |
| Conditions | mL | vp | mL | TCID50 | mL | μg | mL | μg |
| 1% Tergitol 15-S-9, 1 hr | 9.62 × 10¹¹ | 5.28 × 10¹³ | 5.87 × 10¹⁰ | 3.22 × 10¹² | <7 | <0.38 | 68.7 | 3.77 |
| 1% Triton X-100, 1 hr | 7.82 × 10¹¹ | 4.35 × 10¹³ | 4.62 × 10¹⁰ | 2.57 × 10¹² | 17.8 | 0.99 | 88.3 | 4.91 |
| 0.5% OGP, 1 hr | 1.56 × 10¹² | 8.24 × 10¹³ | 3.98 × 10¹⁰ | 2.10 × 10¹² | 154 | 8.1 | <20 | <1.06 |
| 1% Tween 20, 20 mM | 2.58 × 10¹² | 1.53 × 10¹⁴ | 5.51 × 10¹⁰ | 3.27 × 10¹² | 35 | 2.1 | 163.2 | 9.69 |
| PO4 pH 8.0, 2 hr | | | | | | | | |
| 1% Triton X-100, 1 hr | 1.89 × 10¹² | 9.94 × 10¹³ | 4.81 × 10¹⁰ | 2.53 × 10¹² | 15.3 | 0.8 | 94.9 | 5.00 |

hr: hour
vp: viral particle

From Table 8, the data suggests that cell lysis using Tween 20 may show lower efficiency at reducing HC DNA and host cell protein (HCP). Additional small-scale experiments were performed as per Table 9 below to determine the optimal Benzonase digestion conditions to ensure Tween 20 produces HC DNA and HCP levels similar or lower than those resulting from the Triton X-100 process.

TABLE 9
Benzonase Optimization Following Cell Lysis with 1%
Tween 20, 20 mM Na₂HPO₄, pH 8.0, 2 hr Incubation

| Detergent | Concentration | Incubation (37° C., | Benzonase Digestion |
|---|---|---|---|
| Conditions | (v/v) | 120 rpm) | Conditions |
| Triton X-100 (Control) | 1% | 1 hour | 60 minutes, 10 U/mL |
| Tween 20 (QL300) in 20 mM | 1% | 2 hours | 60 minutes, 10 U/mL |
| Sodium Phosphate, pH 8 | | | |
| Tween 20 (QL500) in 20 mM | 1% | 2 hours | 60, 90, 120, 180, 240 |
| Sodium Phosphate, pH 8 | | | minutes, 20 U/mL |

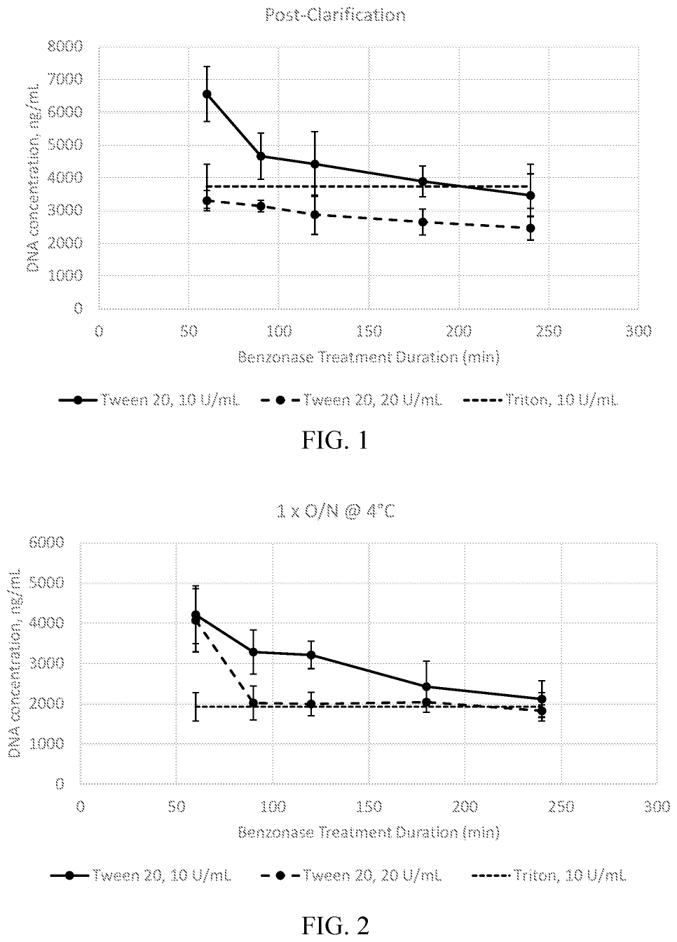

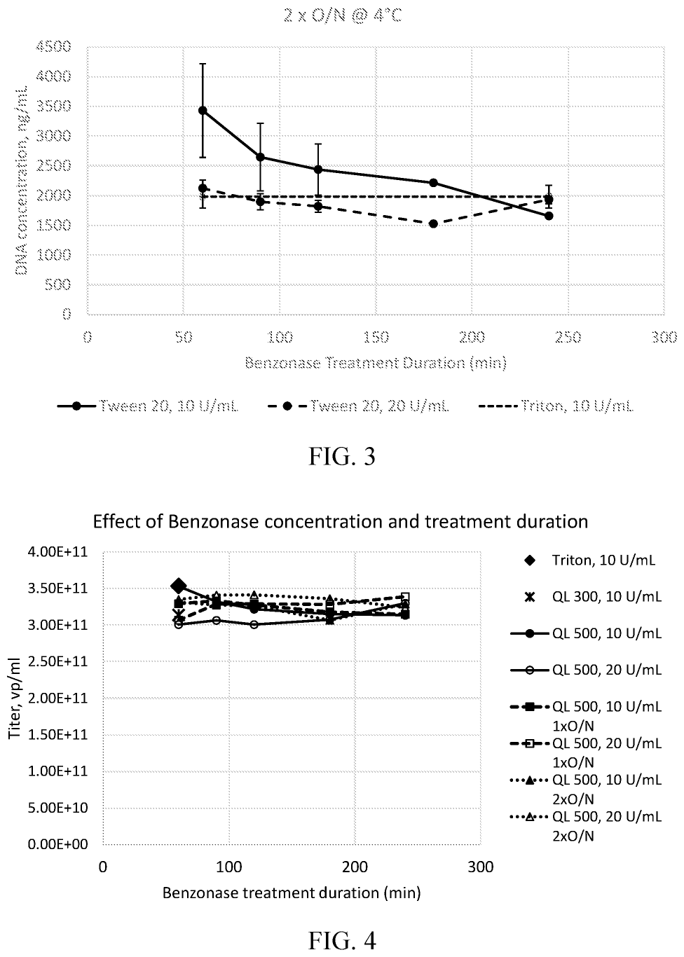

hr: hour As shown in Table 9 and FIGS. 1-3, increasing Benzonase concentration from 10 to 20 U/mL lowered the residual HC DNA levels to those obtained with the Triton X-100 cell lysis. Optimal Benzonase digestion duration, when used at a 20 U/mL concentration, ranges from 90-240 minutes.

As shown in Table 9 and FIGS. 1-4, increasing Benzonase concentration from 10 to 20 U/mL or changing Benzonase treatment duration from 60 to 240 minutes did not negatively impact the total reovirus titer (HPLC), for up to 2 days of overnight storage at 2-8° C. post-clarification.

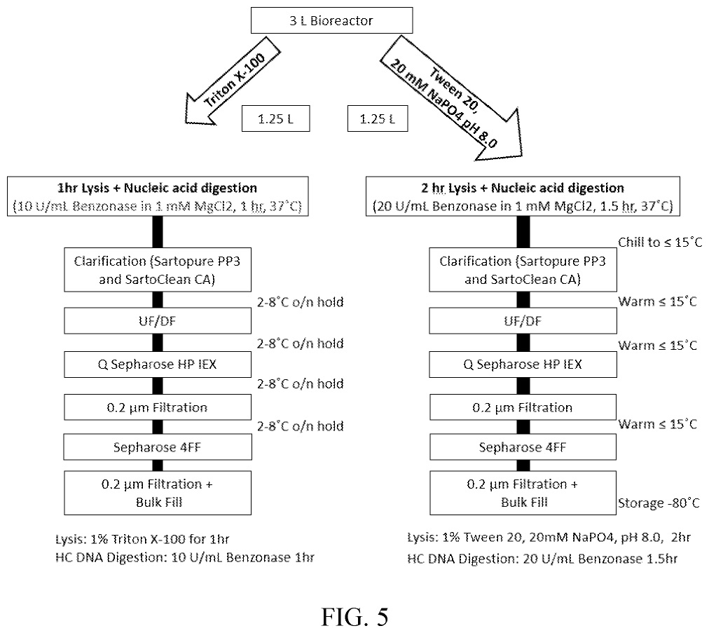

In summary, cell lysis with 1% Tween 20 in 20 mM NaPO₄, pH 8.0 lysis buffer for 2 hr incubation, followed by Benzonase digestion for 1.5 hr at 20 U/mL can be used as shown in the examples and described throughout the application. FIG. 5 shows a schematic of the reovirus purification workflow, including the virus extraction protocol.

The following is an overall summary of the production and extraction of virus as described in the examples. HEK293S cells were thawed and expanded for approximately 6-8 passages until an adequate quantity of cells required to seed the bioreactor at 1.5 L at a target Viable Cell Density of 0.4×10⁶ cells/mL was reached. The bioreactor operating conditions are Temperature=37° C.; Dissolved oxygen=40% air saturation; pH=7.2±0.05; and Agitation rate=150 rpm final (ramp). Once the cell density was reached (between 1.8−2.4×10⁶ cells/mL), the culture is infected. A viral stock was prepared in 24 mL medium for a Multiplicity of Infection (MOI) of 0.5 virus/cell. The virus was added in about 1.5 L of fresh medium to bring the final volume to 3 L. Cell viability was monitored, and cells were harvested either 3 days post-infection (if the viability is below 35%) or at 4 days post-infection. Glucose was added as needed at time of infection.

For harvest, the culture volume of the bioreactor was split into 2 equal volumes that were each distributed in 2×3.5 L Chemap bioreactor vessels. The Triton X-100, 0.91% v/v final was added to one vessel and Tween 20, 1% final in a 20 mM NaPO₄ lysis buffer at pH 8.0 was added to the other vessel. For Triton X-100, after 1 hour of incubation at 37° C. and agitation at 120 rpm, a 500 U/mL Benzonase stock solution in 50 mM MgCl₂ was added for a final concentration of 10 U/mL in 1 mM MgCl₂. After 1 hr of Benzonase digestion, purification of reovirus from the cell cultures was initiated. For Tween 20, 20 mM NaPO₄ pH 8.0, after 2 hours of incubation at 37° C. and 120 rpm, a 1000 U/mL Benzonase solution in 50 mM MgCl₂ was added for a final concentration of 20 U/mL in 1 mM MgCl₂. After 1.5 hr of Benzonase digestion, purification of reovirus from the cell cultures was initiated.

The invention claimed is:

1-41. (canceled)

42. A method of producing reovirus, comprising:
(a) providing a culture of HEK 293 cells which has been infected by reovirus;
(b) contacting the cells with a first buffer comprising Tween 20 and a phosphate and incubating the HEK 293 cells in the presence of the first buffer for a first period of time thereby producing a cell lysate, wherein the incubating occurs at a temperature of about 30° C. to about 40° C. and a pH of about 7.0 to about 8.0;
(c) contacting the cell lysate with a second buffer comprising an endonuclease for a second period of time to degrade nucleic acids of the HEK 293 cells;
(d) removing cell debris by filtration;
(e) concentrating the filtrate by ultrafiltration and diafiltration;
(f) purifying the reovirus by a combination of ion exchange and size exclusion chromatography; and
(g) collecting the reovirus.

43. The method of claim 42, wherein the first buffer comprises 10 mM to 25 mM phosphate.

44. The method of claim 42, wherein the first buffer comprises at least 20 mM phosphate.

45. The method of claim 42, wherein the phosphate is sodium phosphate.

46. The method of claim 42, wherein the first buffer comprises 0.5%, 1.0% or 2.0% (v/v) Tween 20.

47. The method of claim 42, wherein the first period of time is 60, 120, 180 or 240 minutes.

48. The method of claim 42, wherein the second period of time is 60 minutes, 90 minutes, 120 minutes, 180 minutes, or 240 minutes.

49. The method of claim 42, wherein the endonuclease is Benzonase.

50. The method of claim 42, wherein the endonuclease is contacted with the cell lysate at a concentration of 10 to 20 U/mL.

51. The method of claim 42, wherein contacting the cell lysate with the endonuclease occurs at a temperature of 30° C. to 40° C.

52. The method of claim 42, wherein contacting the cell lysate with the endonuclease occurs at a temperature of 35° C. to 39° C.

53. The method of claim 42, wherein incubating the host cells occurs at a temperature of 35° C. to 39° C.

54. The method of claim 42, wherein the second buffer further comprises MgCl₂.

55. The method of claim 42, wherein incubating the host cells in the presence of the first buffer occurs at a pH of 8.0.

56. The method of claim 42, wherein the HEK293 cells are grown in suspension.

57. The method of claim 42, wherein the ion exchange is performed using an anion exchanger.

58. The method of claim 42, wherein the ion exchange is performed prior to the size exclusion chromatography.

59. The method of claim 42, wherein a phosphate buffer is used in the ion exchange.

60. The method of claim 59, wherein the phosphate buffer comprises 100 mM sodium phosphate.

61. The method of claim 42, wherein a phosphate buffer is used in the size exclusion chromatography.

62. The method of claim 61, wherein the phosphate buffer comprises 10 to 15 mM sodium phosphate.

63. The method of claim 42, wherein the size exclusion chromatography is carried out at a pH of about 7.5 to about 8.0.

64. The method of claim 42, wherein the reovirus is a mammalian reovirus.

65. The method of claim 64, wherein the mammalian reovirus is a human reovirus.

66. The method of claim 65, wherein the human reovirus is a serotype 3 reovirus.

67. The method of claim 66, wherein the serotype 3 reovirus is a Dearing strain reovirus.

68. The method of claim 42, wherein the first buffer comprises 1% (v/v) Tween 20.

69. The method of claim 68, wherein the endonuclease is Benzonase and wherein the Benzonase is contacted with the cell lysate at a concentration of 20 U/mL.

70. The method of claim 69, wherein the first period of time is at least 120 minutes and the second period of time is at least 90 minutes.

71. The method of claim 45, wherein the first butter comprises 1% (v/v) Tween 20 and 20 mM sodium phosphate, wherein the pH is 8.0, wherein the first period of time is at least 120 minutes and the second period of time is at least 90 minutes, wherein the endonuclease is Benzonase, and wherein the Benzonase is contacted with the cell lysate at a concentration of 20 U/mL.
